(19) United States
(12) Patent Application Publication
(10) Pub. No.: US 2012/0316211 A1
(43) Pub. Date: Dec. 13, 2012

(54) Diagnostic Methods for Assessing Risk of Chagas Disease and Heart Failure

(75) Inventor: Nisha Jain Garg, League City, TX (US)

(73) Assignee: The Board or Regents of the University of Texas System, Austin, TX (US)

(21) Appl. No.: 13/470,209

(22) Filed: May 11, 2012

Related U.S. Application Data
(60) Provisional application No. 61/518,736, filed on May 11, 2011.

(51) Int. Cl. G01N 33/566; A61P 9/00; A61K 31/04; A61P 33/00; G01N 21/64; A61K 31/4168

(52) U.S. Cl. 514/398; 435/7.1; 435/7.92; 435/7.9; 514/741

(57) ABSTRACT

Provided herein are methods of detecting evidence of Chagas disease in a biological sample, comprising the step of measuring the presence of at least one protein selected from the group consisting of gelsolin, myosin light chain 2, vimentin, myosin heavy chain 11, vinculin, and plasminogen in said sample, wherein significantly elevated levels of the protein is a biomarker for the presence or severity of Chagas disease.

### PRIORITY

This application claims priority to U.S. Provisional Patent Application No. 61/518,736 filed May 11, 2011, which is incorporated herein by reference in its entirety.

### STATEMENT REGARDING FEDERALLY FUNDED RESEARCH

This invention was made with government support under Grant Numbers HL08823002 and HL094802 awarded by the National Institutes of Health. The government has certain rights in the invention.

### BACKGROUND

The present invention relates to the field of human and animal health and in particular to diagnostic methodologies useful in determining the risk and severity of Chagas disease.

Chagas disease continues to pose a serious threat to health in Latin America and Mexico, and is an emerging parasitic disease in developed countries including the U.S. According to World Health Organization reports, the overall prevalence of human Trypanosoma cruzi infection is at ˜16-18 million cases, and ˜120 million people, i.e. 25% of the inhabitants of Latin America, are at risk of infection (World Health Org. (2006) Report of the Scientific Working Group on Chagas Disease; WHO. (2010) Chagas disease: control and elimination. Report of the secretariat). It is estimated that >300,000 infected patients live in the United States (Bern and Montgomery, (2009) Clin Infect Dis, 49, e52-54). Of those infected, 30-40% progress to an irreversible cardiomyopathy several years following infection, which results in considerable morbidity and mortality (World Health Org. (2006) Report of the Scientific Working Group on Chagas Disease). Moreover, no vaccines are available. Benznidazole, the available drug therapy, is effective in controlling parasitemia in acutely infected individuals (Inglessis et al. (1998) Arch Inst Cardiol Mex, 68, 405-4104; Bastos et al. (2010) PLoS Negl Trop Dis, 4, e711-5); its efficacy, however, in arresting or reversing disease progression in chronically infected patients is not clearly established (Urbina, (2010) Acta Trop, 115, 55-686; Maya, et al. (2010) Biol Res, 43, 323-331). It is crucial that molecular markers are identified that could allow classification of disease state and detection of asymptomatic individuals who are at risk of developing chagasic cardiomyopathy.

The red and white blood cells are dynamic components of the circulatory system and interact with all cells, tissues, and organs, specifically the heart. It is, therefore, possible that the pathologic processes during the development of Chagas disease would cause characteristic changes in the circulating proteins (e.g., level, oxidation) and generate a detectable, disease-specific molecular phenotype. With long-term cardiac injury, as noted in a majority of chronic chagasic patients (Marin-Neto et al., (2007) Circulation, 115, 1109-1123; Saravia et al. (2011) Arch Pathol Lab Med, 135, 243-248), the progression of disease severity is presented by an increasing order of cell death, heart decompensation, and a drop in cardiac output, leading to heart failure (Rassi et al. (2006) N Engl J Med, 355, 799-808; Rassi et al. (2010) Lancet, 375,1388-1402). Cell death during this process may result in the sustained release of cardiac proteins in the peripheral system. These cardiac proteins and their disease-dependent modified forms in plasma are potential cardiac-specific biomarkers (Sabatine et al. (2002) Circulation, 105, 1760-1763; Stanley et al. (2004) Dis Markers, 20, 167-178).

Several studies have implicated the role of central and peripheral inflammatory mechanisms and oxidative stress in Chagas disease (Maya et al., (2010) Biol Res, 43, 323-331; Zacks et al., (2005) An Acad Bras Cienc, 77, 695-715; Gupta et al., (2009) Interdiscip Perspect Infect Dis, 2009,190354). In experimental animal models and human patients, parasite persistence results in consistent activation of inflammatory responses and leads to the development and/or propagation of pathological lesions in the heart (Schijman et al., (2004) Am J Trop Med Hyg, 70, 210-220; da Silveira et al., (2005) Parasitology, 131, 627-634; Wen et al., (2006) Am J Pathol, 169, 1953-1964). In other studies, myocardial production of reactive oxygen species (ROS) due to mitochondrial dysfunction of the electron transport chain and release of electrons to molecular oxygen has been found to be the major source of oxidative stress in chagasic hearts (Wen et al., (2004) Free Radic Biol Med, 37, 1821-1833; Wen et al., (2008) Microbes Infect, 10, 1201-1209; Wen and Garg, (2008) J Bioenerg Biomembr, 40, 587-598; Wen and Garg, (2010) Antioxid Redox Signal, 12, 27-37). Recent studies demonstrated that an increase in myocardial oxidative damage correlated with an antioxidant inefficiency and cardiac dysfunction. Further, treatment of infected animals with an antioxidant was effective in arresting the oxidative cardiac pathology (Wen et al., (2006) Am J Pathol, 169, 1953-1964) and preventing the loss of cardiac LV function in chronic hearts (Wen et al., (2010) J Am Coll Cardiol, 55, 2499-2508), thus, establishing the pathological significance of oxidative overload in Chagas disease.

Blood serves as a useful tissue capable of detecting and responding to the changes induced in the body during the course of T. cruzi infection and disease development. The changes in immune response, oxidative stress, and antioxidant imbalance are detectable in peripheral blood of infected mice (Wen et al., (2008) Microbes Infect, 10, 1201-1209), and, notably, a strong positive correlation was detected for the disease state-specific changes in the heart-versus-blood level of oxidative stress markers and antioxidants (e.g. glutathione peroxidase, glutathione, manganese superoxide dismutase) (Wen et al., (2008) Microbes Infect, 10, 1201-1209). Distinct plasma protein-nitrotyrosylation profiles have also been documented in acutely- and chronically-infected chagasic animals (Dhiman et al., (2008) Am J Pathol, 173, 728-740). These studies, along with documentation of oxidative overload in chagasic humans (Wen et al., (2006) Free Rad Biol Med, 41, 270-276; de Oliveira et al., (2007) Int J Cardiol, 116, 357-363), support the idea that characterization of plasma proteomes will be useful in identifying the molecular mechanisms that are disturbed during the progression of Chagas disease.

Thus, there is a recognized need in the art for diagnostic methods of Chagas disease development in humans and other animals. The present invention fulfills this long-standing need and desire in the art.

### SUMMARY

Certain embodiments are directed to a method of detecting evidence of chagasic cardiomyopathy in a biological sample, comprising the step of measuring the level or presence of at least one protein selected from the group consisting of gelsolin (GSN), myosin light chain 2 (MYL2), vimentin (VIM), myosin heavy chain 11 (MYH11), vinculin (VCL), and plasminogen (PLG) in the sample. In an undiagnosed subject, the levels of one or more of the proteins can be indicative of T. cruzi infection. In a subject already diagnosed with T. cruzi infection, the protein levels can be indicative of the severity of disease, e.g., the risk of developing, or the stage of chagasic cardiomyopathy in the subject. Elevated levels of gelsolin (GSN), myosin light chain 2 (MYL2), vimentin (VIM), myosin heavy chain 11 (MYH11), vinculin (VCL), and/or plasminogen (PLG) biomarkers in the samples is indicative of T. cruzi infection, Chagas disease, and/or chagasic cardiomyopathy in the subject.

Certain aspects of the invention are directed to assessment of a subject for risk of developing chagasic cardiomyopathy. Levels of biomarkers are measured and these measurements indicate whether a subject is at risk of developing cardiomyopathy. In certain aspects, the biomarkers measured are VIM, GSN, MYL2, MYH11, VCL, and PLG. In a further aspect, levels of VIM are measured in combination with one or more of GSN, MYL2, MYH11, VCL, or PLG. In a further aspect, levels of GSN are measured in combination with one or more of VIM, MYL2, MYH11, VCL, or PLG. In still a further aspect, levels of MYL2 are measured in combination with one or more of VIM, GSN, MYH11, VCL, or PLG. In certain aspects, levels of MYH11 are measured in combination with one or more of VIM, GSN, MYL2, VCL, or PLG. In further aspects, levels of VCL are measured in combination with one or more of VIM, GSN, MYL2, MYH11, or PLG. In still a further aspect, levels of PLG are measured in combination with one or more of VIM, GSN, MYL2, MYH11, or VCL.

In certain aspects, the methods include treating a subject identified as (a) having T. cruzi infection, (b) at risk of developing chagasic cardiomyopathy, or (c) diagnosed with chagasic cardiomyopathy. Treatments can include anti-trypanosome treatments, or preventive or therapeutic treatments for cardiomyopathy, or a combination of both.

Certain aspects are directed to methods for screening blood comprising measuring the levels of one or more of VIM, GSN, MYL2, MYH11, VCL, and PLG proteins. An increased level of one or more of these proteins is indicative of Trypanosome contamination. In certain aspects blood is screened prior to or during banking. In a further aspect, the methods can further comprise conducting confirmatory testing if the levels of one or more of the biomarkers are elevated.

Certain embodiments are directed to serodiagnostic kits for determining whether a subject is infected with Trypanosoma cruzi and/or staging the severity of Chagas disease, said kit comprising: (a) an antibody directed against VIM, GSN, MYL2, MYH11, VCL, and/or PLG, wherein said antibody is linked to a reporter molecule; (b) a buffer; and c) a reagent for detection of the reporter molecule.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The term “antigen” as used herein is defined as a compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein.

The term “antibody” as used herein includes immunoglobulin molecules and immunologically active portions of immunoglobulin molecules, i.e., molecules that contain an antigen binding site that specifically binds (immunoreacts with) an antigen. A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Specific, non-limiting examples of binding fragments encompassed within the term antibody include (i) a Fab fragment consisting of the V_{L}, V_{H}, C_{L} and C_{H1} domains; (ii) an F_{d} fragment consisting of the V_{H} and C_{H1} domains; (iii) an Fv fragment consisting of the V_{L} and V_{H} domains of a single arm of an antibody, (iv) a dAb fragment (Ward et al., Nature 341:544-546, 1989); and (vi) a F(ab′)₂ fragment. Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Falkner et al., Nature 298:286, 1982; Morrison, J. Immunol. 123:793, 1979; Morrison et al., Ann Rev. Immunol 2:239, 1984).

The term “animal” as used herein refers to living multi-cellular vertebrate organisms, a category that includes, for example, mammals and birds. The term mammal includes both human and non-human mammals. Similarly, the term “subject” includes both human and veterinary subjects. The term mammal includes dogs, cats, cattle, horses, goats, sheep, and other domesticated mammals, as well as non-domesticated mammals. In particular embodiments, the animal is a triatome (the insect vector of T. cruzi) host, e.g., a human, opossum, raccoon, armadillo, squirrel, rat, or mouse.

The term “diagnostic” refers to identifying the presence or nature of a pathologic condition. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis.

“Prognosis” is a probability that a pathologic condition will develop (e.g., result in additional sequelae) or progress (e.g., increase in severity).

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

The following abbreviations may be used herein: 2D-GE: Two-dimensional gel electrophoresis; BZ: Benznidazole; DNPH: 2,4-dinitrophenylhydrazine; GSN: Gelsolin; IPA: Ingenuity pathways analysis; IPG: Immobilized pH gradient; MYL2: Myosin light chain polypeptide 2, cardiac specific; PBN: Phenyl-α-tert-butyl nitrone; RA: Acutely infected rats; RAP: Acutely infected/PBN-treated rats; RC: Chronically infected rats; RCB: Chronically infected/BZ-treated rats; RCP: Chronically infected/PBN-treated rats; RCPB: Chronically infected/PBN+BZ-treated rats; RN: Normal rats; ROS: Reactive oxygen species; VIM: Vimentin.

### DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1H show data derived from Sprague Dawley rats that were infected with T. cruzi and treated with phenyl-α-tert-butyl nitrone (PBN, antioxidant). Some rats were treated with benznidazole (BZ, anti-parasite drug) at the end of the acute infection phase. Plasma samples were collected during acute infection (25 dpi) and chronic disease (150 dpi) phases, and depleted of high-abundance proteins to enrich the detection of low-abundance proteins. Shown are representative, two-dimensional gel electrophoresis (2D-GE) patterns of depleted plasma from 10 normal rats (FIG. 1A), acutely infected rats (FIG. 1B) treated with PBN (FIG. 1C), and chronically infected rats (FIG. 1D) treated with PBN (FIG. 1E), BZ (FIG. 1F), or PBN and BZ (FIG. 1G). FIG. 1H is a bar graph showing the average number of protein spots identified in depleted plasma from each experimental group. Proteins altered in expression by >2-fold were identified by mass spectrometry (listed in Tables 1a-1b).

FIGS. 2A-2J show an expanded view of the 2D-gel images for selected plasma proteins that were altered in expression (>2-fold) in response to T. cruzi infection, disease state, and/or treatment with PBN and BZ. The number on the left side represents the spot number identified on 2D-gels. The gene name of identified proteins, determined by mass spectrometry/BLAST analysis, is shown on the right side. The detailed gene/protein names, along with spot numbers, are listed in Tables 1a-1b. Arrows mark the disease- or treatment-specific change in plasma proteins in chagasic rats. Intensity of color was indicative of the extent of change in the protein level in chagasic plasma.

FIGS. 3A-3H show protein carbonyls, which are an indicator of protein oxidation, detected in plasma of T. cruzi infected rats. Depleted plasma samples from normal and T. cruzi infected rats (±PBN/BZ) were subjected to 1^{st}-dimension isoelectric focusing on 11-cm, linear pH 5-8 immobilized pH gradient (IPG) strips. Strips were incubated with dinitrophenylhydrazine (DNPH) to derivatize carbonyl proteins, and 2^{nd}-dimension separation was carried out by SDS-PAGE on 8-10% gradient gels. FIGS. 3A-3G: Shown are the representative images of Western blotting with anti-DNP antibody. FIG. 3H: The bar graph shows average number of carbonylated protein spots identified in the plasma of each experimental group. Carbonyl proteins were identified by mass spectrometry.

FIGS. 4A-4D show that western blotting confirmed the differential expression profile of selected proteins in chagasic rat plasma. FIGS. 4A-4C: Whole plasma from normal and infected rats (±PBN/BZ) were resolved by SDS gel electrophoresis, and membranes were probed using anti-gelsolin (Aa), anti-MYL2 (Ba) and anti-vimentin (Ca) antibodies. The expanded view of the corresponding spot for gelsolin, MYL2, and vimentin from 2D-gels is presented in panels Ab, Bb, and Cb, respectively. Coomassie-stained gel image demonstrates equal loading of all samples (FIG. 4D).

FIGS. 5A-5D show that expression profiles of GSN, MYL2 and VIM are altered in chagasic human patients. Human plasma samples were obtained from patients graded according to Chagas disease (CD) severity as CD0, CD1, CD2, and CD3. Samples from normal (N) subjects were utilized as controls. Plasma samples were resolved by SDS-PAGE and Western blotting performed by using anti-gelsolin (FIG. 5A), anti-MYL2 (FIG. 5B), and anti-vimentin (FIG. 5C) antibodies. Coomassie-stained gel image demonstrates the equal loading of all samples (FIG. 5D).

FIGS. 6A-6E show an example of optimization of 2D-GE for profiling rat plasma proteome. (FIG. 6A) Normal rat plasma samples were collected on K₃EDTA, and enriched for low-abundance proteins by using IgY immune-affinity columns that bind to and remove the high-abundance proteins. Abbreviations include—M: Molecular weight marker, WP: Whole plasma, DP: Plasma depleted of high-abundance proteins, BP: Binding proteins removed by the IgY columns. The high-abundance proteins (right-side margin) were predicted based on molecular weight. Shown are representative SYPRO Ruby stained 2D-gel images of whole plasma (FIG. 6B), high-abundance binding fraction (FIG. 6C), and depleted plasma (FIGS. 6D and 6E). The depleted plasma proteome resolution was enhanced when IEF first dimension was performed on IPG strips pH 5-8 range (FIG. 6E).

FIGS. 7A-7C illustrate distribution analysis of protein spots differentially expressed in response to T. cruzi infection. Sprague Dawley rats were infected with T. cruzi and treated with phenyl-α-tert-butyl nitrone (PBN) and benznidazole (BZ) as described below. Plasma samples were collected at day 25 (acutely infected) and 150 (chronically infected) post-infection. Shown are the percentage (FIG. 7A) and absolute number (FIG. 7C) of differentially expressed protein spots detected by 2D-GE analysis of plasma samples from normal rats (RN), rats that were acutely infected (RA), rats acutely infected and treated with PBN (RAP), chronically infected rats (RC), and chronically infected rats that were treated with PBN (RCP), BZ (RCB), or PBN and BZ (RCPB). The percentage of differentially expressed plasma proteins associated with disease state and/or treatment is shown in (FIG. 7B).

FIGS. 8A-8J are an expanded view of the 2D-gel images for plasma proteins that were differentially expressed in response to T. cruzi infection (±PBN/BZ). Shown are proteins down regulated (FIG. 8A) or up regulated (FIG. 8B) in response to T. cruzi infection, and specifically altered in plasma of acutely (FIG. 8C) and chronically (FIG. 8D) infected rats. Plasma proteins that were stimulated in response to PBN treatment in acutely (FIG. 8E) and chronically (FIG. 8F) infected rats, or normalized by PBN in infected rats (FIG. 8G) are shown. Shown in FIGS. 8H-8J are plasma proteins that were down regulated (FIG. 8H) or up regulated (FIG. 8I) in BZ-treated and PBN/BZ-treated (FIG. 8J) chronically infected rats. The number on the left-side margin represents the spot number identified on 2D-gels and listed in Tables 1a-1b. The gene identity, determined by mass spectrometry/BLAST analysis, is shown in the right-side margin. Arrows mark the disease- or treatment-specific increase (or decrease) in protein levels in chagasic plasma. On top of each panel, sequential plasma pattern of RN, RA, RAP, RC, RCP, RCB, and RCPB are marked.

FIGS. 9A-9D show an example of validation studies on the expression profile of selected proteins in chagasic sera. Whole sera samples from normal (n=20) and chagasic (n=35) subjects were subjected to ELISA analysis by using antibodies against vinculin (VCL) (FIG. 9A), myosin light chain 2 (cardiac specific, MYL2) (FIG. 9B), Vimentin (VIM) (FIG. 9C), and plasminogen (PLG) (FIG. 9D). Standard deviation for triplicate observations for each sample was <12%. Shown is box plot of ELISA data, graphically depicting the values for seronegative and seropositive groups. The horizontal lines of the box (bottom to top) depict the lower quartile (Q1, cuts off lowest 25% of the data), median (Q2, middle value), and upper quartile (Q3, cuts off the highest 25% of the data). The lower and upper whiskers depict the smallest and largest non-outlier observations, respectively, and solid dots represent the outliers. The spacing between the different parts of the box indicates the degree of dispersion (spread).

### DESCRIPTION

The present invention developed a comprehensive plasma proteome profile during T. cruzi infection and disease development in a rodent model of Chagas disease. Parasite persistence and oxidative damage in the heart are known to be of pathological significance during Chagas disease (Zacks et al., (2005) An Acad Bras Cienc, 77, 695-715; Gupta et al., (2009) Interdiscip Perspect Infect Dis, 2009,190354). Infected rats treated with an anti-parasite drug (BZ) and/or antioxidant (PBN) showed that the beneficial effects of these treatments in controlling parasite- and oxidative stress-induced pathology, respectively (Wen et al., (2006) Am J Pathol, 169,1953-1964; Wen et al., (2010) J Am Coll Cardiol, 55, 2499-2508), are reflected in a plasma proteome profile of chagasic rats. The inventor identified 92 proteins that were differentially expressed or oxidized in chagasic plasma. Functional analysis allocated a majority of these proteins to inflammation/immunity and lipid metabolism categories, and to molecular pathways associated with cardiovascular dysfunction, e.g., myocardial infarction, hypertrophy, and fibrosis, and pulmonary embolism and hypertension. Some proteins in chagasic rats treated with PBN and/or BZ were allocated to curative pathways (immune regulation and cardiac remodeling). The 2D-GE results were validated by Western blotting. It was demonstrated that the disease-associated increased expression of GSN and VIM, and release of cardiac MYL2 in the plasma of chagasic rats was normalized by PBN/BZ treatment. Increased plasma levels of GSN, MYL2, and VIM were directly correlated with the severity of cardiac disease in human chagasic patients. This is the first study demonstrating that the plasma oxidative and inflammatory response profile and plasma detection of cardiac proteins parallel the pathologic events contributing to Chagas disease development. These findings have utility in diagnosing disease severity and designing suitable therapy for management of human chagasic patients.

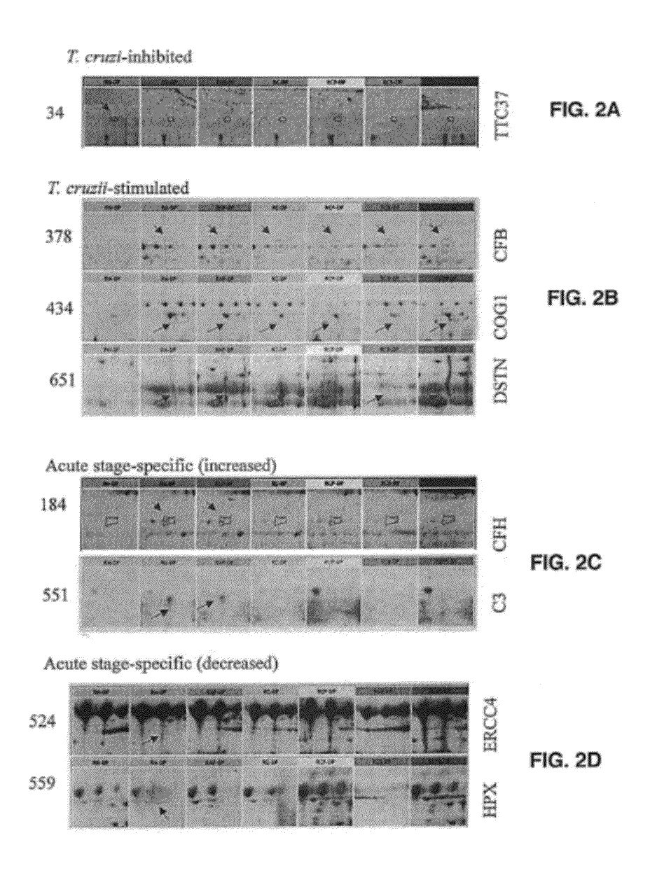

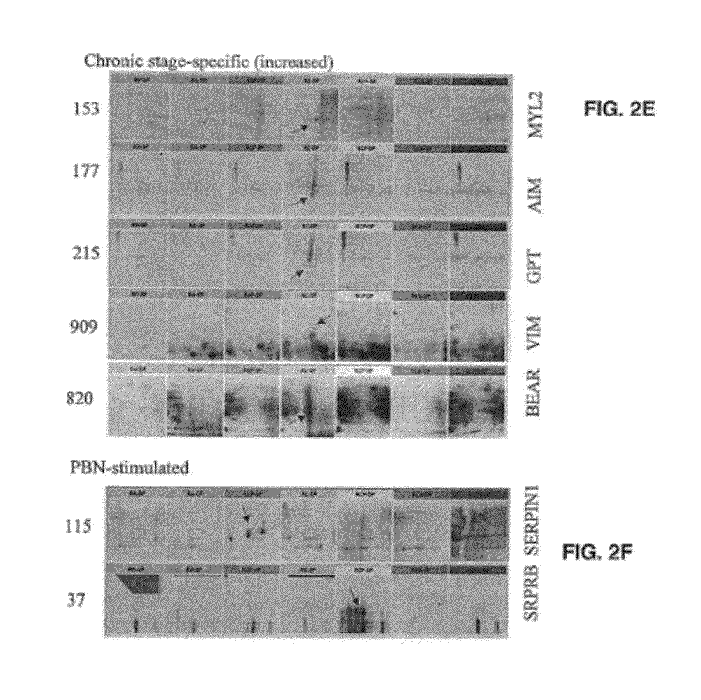

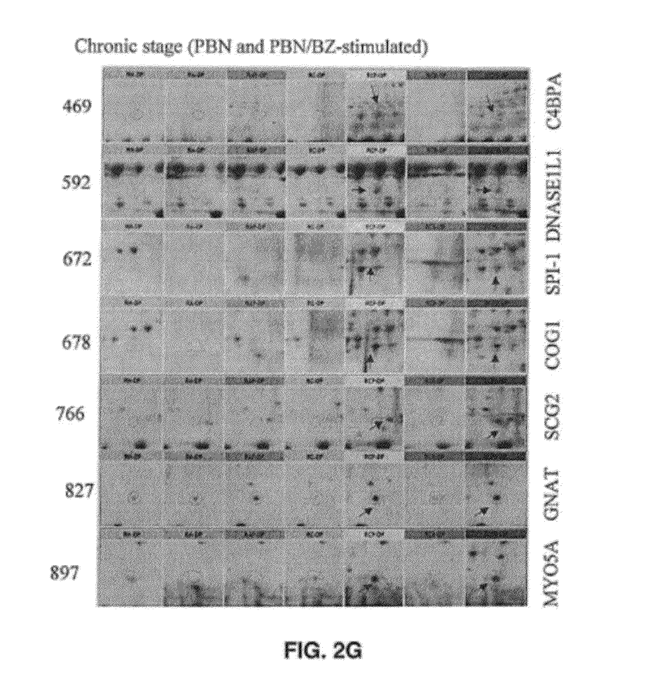

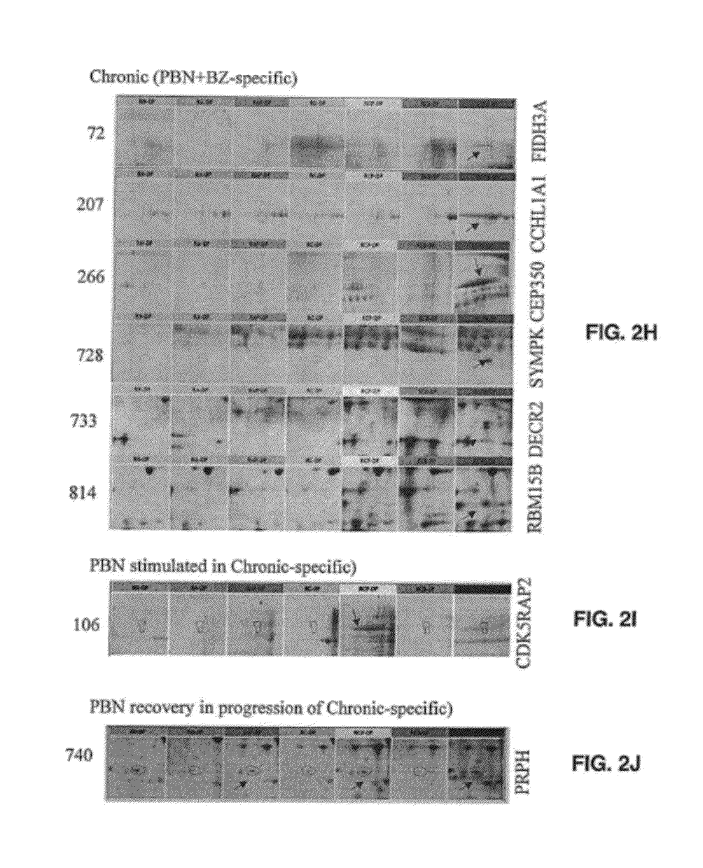

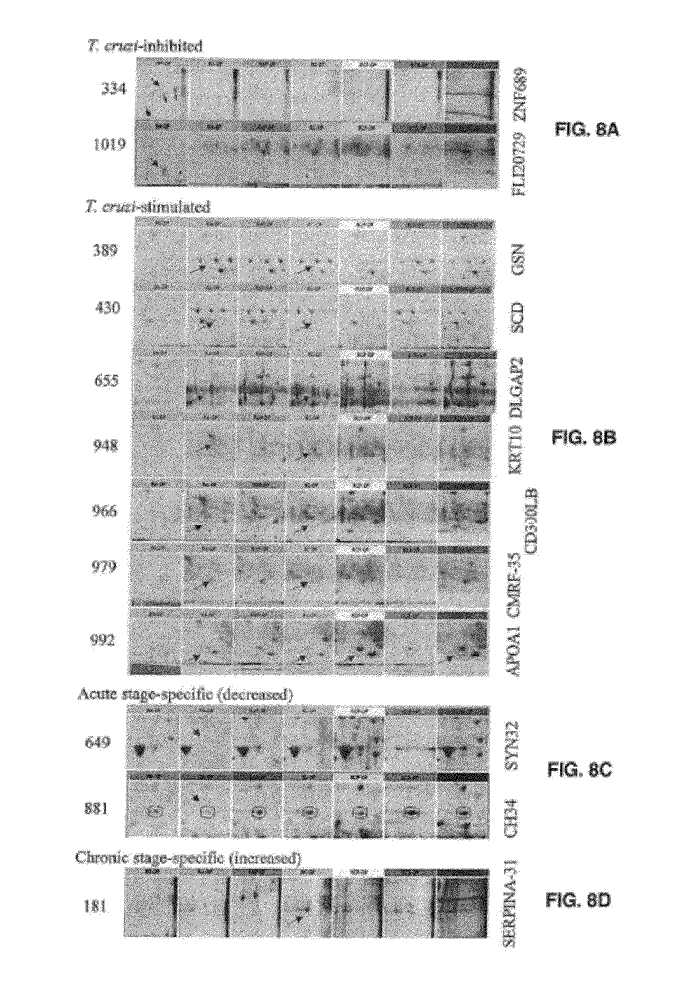

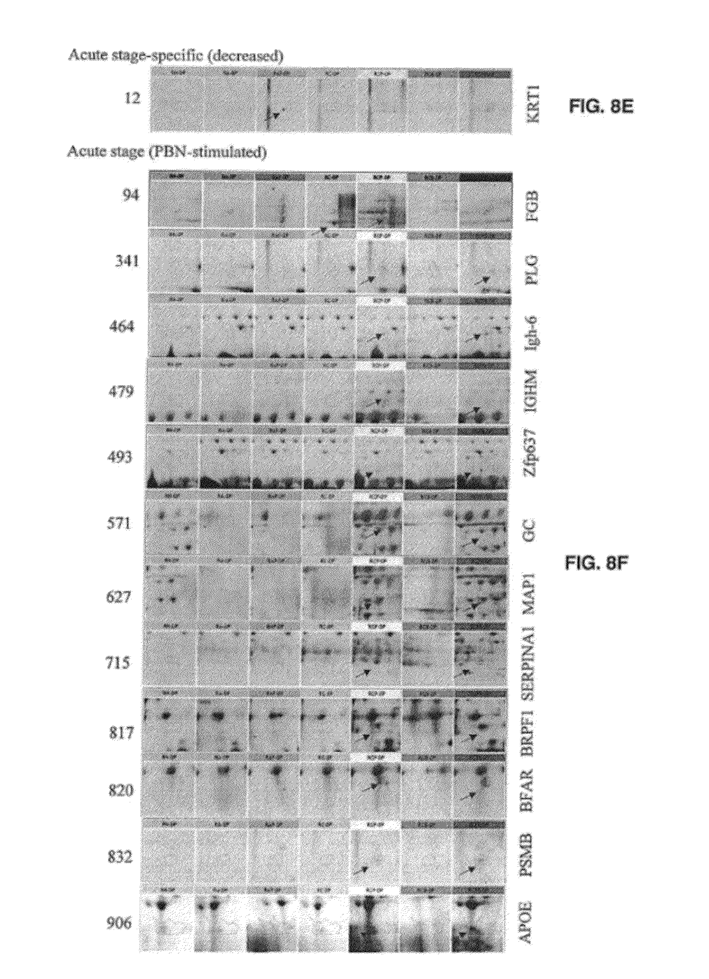

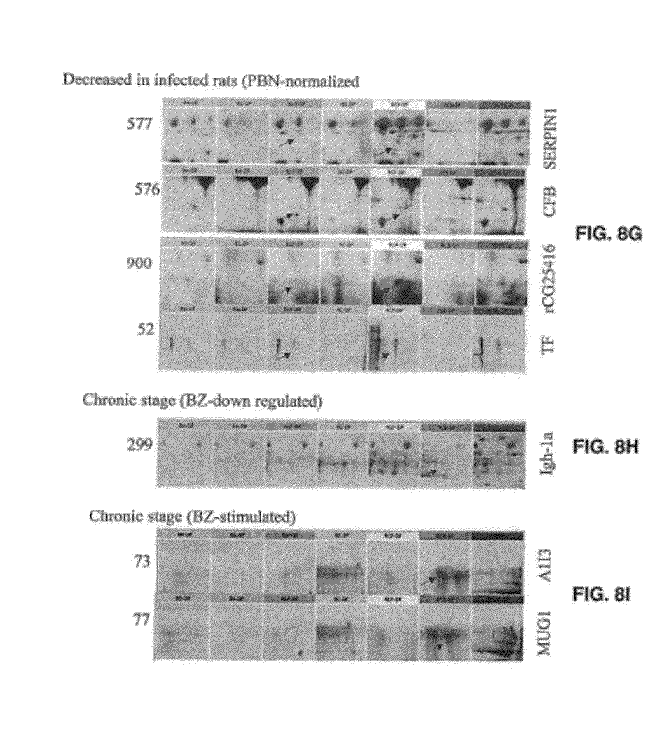

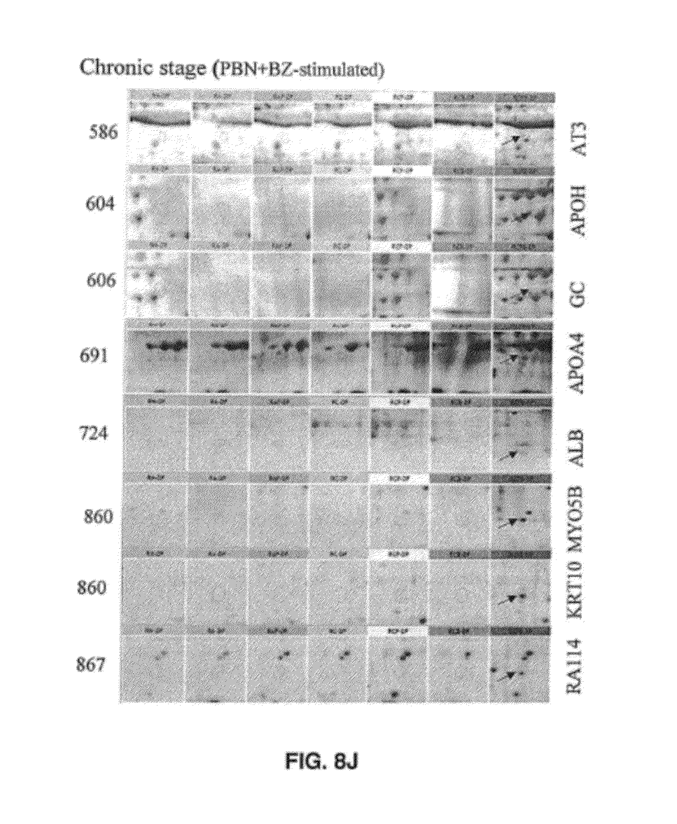

Inflammation/immune response. Ingenuity Pathway Analysis (IPA) is a highly curated and comprehensive software used for the integration of proteins into networks and pathways with biological meaning (Thomas and Bonchev, (2010). Hum Genomics, 4, 353-360). Network analysis of the plasma proteome profile of chagasic rats identified four major sub-networks linked to host response to T. cruzi infection and disease development (Table 2). The maximal numbers of the differentially expressed plasma proteins (19 proteins) in chagasic rats were associated with antigen presentation and inflammatory response category (Table 2) and indicators of persistent inflammation, known to be of pathological significance in Chagas disease (Dhiman et al., (2009) Clinical and Vaccine Immunology, 16, 660-666; Tanowitz et al., (2009) Prog Cardiovasc Dis, 51, 524-539; Junqueira et al., (2010) Expert Rev Mol Med, 12, e29). Following functional analysis of the inflammation-associated proteins, 12 proteins (APOA1, APOE, C3, CFB, CFH, FGB, GSN, KRT10, PLG, SCG2, SERPINA1, and SERPINC1) were identified as being involved in immune cell trafficking and cell movement of leukocytes, granulocytes, phagocytes, neutrophils, and dendritic and antigen presenting cells. Some of the differentially expressed proteins in inflammation category were associated with activation (APOE, C3, CFH, GC and PLG), chemotaxis (C3, SCG2, SERPINA1, SERPINC1), and infiltration of leukocytes (APOA1, APOE, C3, PLG, KRT10) and neutrophils (C3, CFB, CFH, PLG). Up regulation of APOE, APOA1, APOH, GC and PLG in chagasic plasma was indicative of activation, binding and accumulation of macrophages in the disease state. Of the 19 differentially expressed inflammation-associated proteins, 11 proteins (APOE, C3, CFB, CFH, GSN, KRT10, SCG2, SERPINA1, SERPINC1, GC, and PLG) were carbonylated in chagasic plasma. PBN treatment prevented the oxidative modification of five of these proteins (i.e., CFB, CFH, SERPRINA1, SERPINC1, and PLG). Interestingly, PBN treatment also normalized or regulated the expression of several inflammation-associated proteins, including APOH, APOE, CFH, PLG and SCG2 in acutely infected rats and APOH, GC, GSN and PLG in chronically infected rats; while the expression level of CFB, GSN, C3, and SERPINC 1 was partly regulated by PBN in infected rats (Tables 1a-1b, FIGS. 2A-2J and FIGS. 8A-8J). Other proteins (APOE, C3, KRT3, and SCG2) were exposed to oxidation due to T. cruzi-induced, acute oxidative stress, but were normalized in expression and oxidation during the chronic phase.

Treatment of rats with anti-parasite drug (BZ) was not effective in preventing protein carbonylation. These observations indicate that oxidative stress plays an important role in modulating the host immune response against T. cruzi. ROS elicit inflammatory cytokines (e.g. TNF-α, IFN-γ, IL-1α) in cardiomyocytes infected by T. cruzi (Gupta et al., (2009) Free Radio Biol Med, 47,1414-21; Ba et al., (2010) J Biol Chem, 285, 11596-606). Inflammatory pathology was controlled in chronically infected experimental animals and human patients by enhancing the antioxidant status, which was also beneficial in preserving the cardiac function during Chagas disease (Wen et al., (2006) Am J Pathol, 169, 1953-1964; Wen et al., (2010) J Am Coll Cardiol, 55, 2499-2508; Ba et al., (2010) J Biol Chem, 285, 11596-606; Souza et al., (2010) Mem Inst Oswaldo Cruz, 105, 746-751). Recent observations indicate that the mitochondrial release of ROS due to electron transport chain dysfunction and enhanced release of electrons to molecular oxygen is the primary source of oxidative stress in the heart (Wen and Garg, (2008) J Bioenerg Biomembr, 40, 587-598).

Lipid Metabolism. Seventeen of the differentially expressed proteins in chagasic plasma, i.e., AFM, C4BPA, CACNAID, DLGAP2, GC, KNG1, MUG1, MYLPF, MY05A, MY05B, PRPH, PZP, RAI14, and SCG2, were allotted to the lipid metabolism/molecular transport/small molecule biochemistry category (Table 2) and functionally linked by IPA network analysis to lipid, fatty acid, and carbohydrate metabolism. A majority of the proteins in this category, i.e., ALB, APOA1, APOA4, APOE, APOH, C3, GC, GNAQ, MY05A, PLG, SCD2, SERPINA1, SERPINC1 and VIM, were linked to the synthesis, metabolism, transport, and modification of lipids and fatty acids, and to uptake and release or efflux of lipids, eicosanoids, and cholesterol. PBN/BZ-treated/infected rats exhibited normalization in the expression of 13 of the proteins linked to lipid/fatty acid metabolism (Tables 1a-1b). These data provide the first indication that lipid/fatty acid metabolism is dysregulated and of pathologic significance in Chagas disease. The observation of increased expression of CEP350 in chagasic rat plasma provides clues to the pathologic mechanism involved in altered lipid/fatty acid metabolism during Chagas disease. CEP350 is a large centrosome-associated protein with a CAP-Gly domain typically found in cytoskeleton-associated proteins (Yan et al., (2006) Mol Biol Cell, 17, 634-44; Patel et al., (2005) J Cell Sci, 118, 175-86). CEP350 interacts with other centrosomal proteins (e.g. FGFR1) and has been implicated in the mechanisms underlying microtubule anchoring and organization at the centrosome (Yan et al., (2006) Mol Biol Cell, 17, 634-44). Interestingly, CEP350 is also shown to alter the activity and sub-cellular compartmentalization of members of the peroxisome proliferator-activated receptors family (PPARα, PPARβ/δ, and PPARγ) (Patel et al., (2005) J Cell Sci, 118, 175-86) that heterodimerize with retinoid X receptors (RXRs) to function as transcription factors, and play essential roles in the regulation of cell differentiation and lipid/fatty acid metabolism (Qi et al., (2000) Cell Biochem Biophys, 32 Spring, 187-204; Szatmari et al., (2007) Blood, 110, 3271-80). Besides CEP350, SERPINs and GPT that were up regulated in chagasic plasma and function in inflammatory response/tissue remodeling and amino acid metabolism, respectively, also belong to the network of proteins regulated by PPARs (Carter and Church, (2009) PPAR Res, 2009, 345320; Rakhshandehroo et al., (2010) PPAR Res, 2010).

Cardiovascular disease-associated proteins. Twenty-four of the differentially expressed proteins, i.e., AFM, ALB, APOAI, APOA4, APOE, APOH, C3, CFB, CFH, FGB, GC, GNAQ, GSN, HPX, ITIH4, KNG1, MY05A, PLG, SCD2, SCG2, SERPINA1, SERPINC1, SERPINF1, and VIM, were linked to cardiovascular function, skeletal and muscular disorders, and cardiovascular diseases (Table 2). Of these, nine proteins (APOA1, APOE, APOH, C3, KNG1, PLG, SCG2, SERPINC1, and SERPINF1) play a functional role in the proliferation of endothelial cells, while several others correlate with angiogenesis (APOE, APOH, PLG, SCG2, SERPINC 1, and SERPINF1), thrombosis or thromboembolism (APOE, APOH, CFB, GNAQ, PLG, and SERPINC1), myocardial ischemia and infarction (ALB, C3, FGB, GSN, PLG, APOA1, APOE, PLG, and SERPINC1), and atherosclerosis (APOA1, APOA4, APOE, and PLG) (Diez et al., (2010) Mol Biosyst, 6, 289-304). The latter findings indicate that endothelial cell dysfunction plays a role in the progression of Chagas disease and demonstrate that the plasma proteome profile is a useful indicator of clinical disease status.

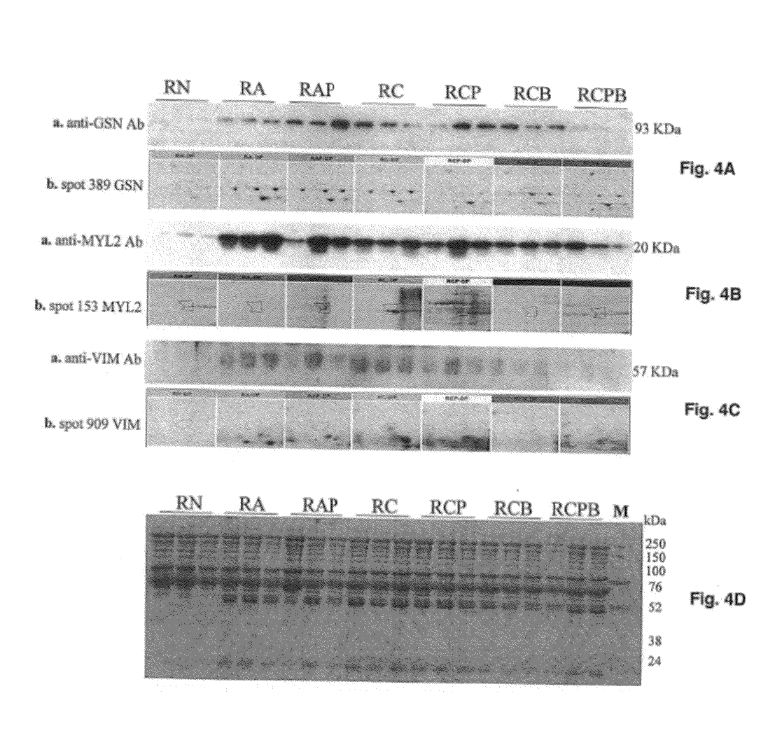

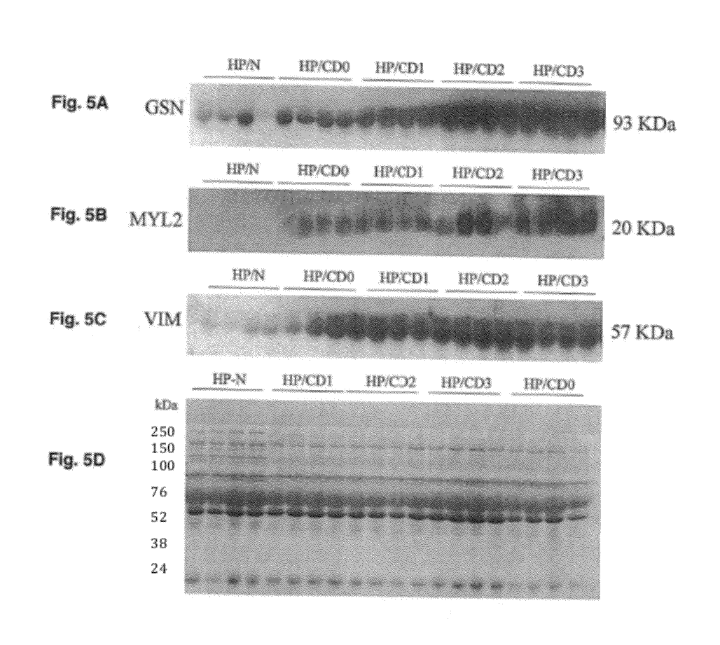

Biomarkers of Chagas disease. An objective of the studies described below was to identify the diagnostic biomarkers of Chagas disease. Western blot analysis using antibodies specific to GSN, MYL2, and VIM validated the 2D-GE plasma profile of chagasic rats and demonstrated that GSN, MYL2, and VIM are indeed increased in the plasma of chagasic rats (FIG. 4). There was a direct correlation in plasma levels of GSN, MYL2, and VIM and disease severity in human chagasic patients (FIG. 5). Further, PBN/BZ-mediated control of cardiac pathology and preservation of heart contractile function in chagasic rats (Wen et al., (2006) Am J Pathol, 169, 1953-64; Wen et al., (2010) J Am Coll Cardiol, 55, 2499-508) was associated with normalized plasma levels of GSN, MYL2, and VIM similar to that noted in normal controls (FIG. 4). These findings indicate the importance of GSN, MYL2, and VIM as protein biomarkers of Chagas disease.

Pathologic significance of GSN, MYL2, and VIM in cardiovascular diseases. Besides their importance as diagnostic markers in Chagas disease observed herein, GSN, MYL2, and VIM play a significant role in heart disease. GSN is an actin-binding protein, and a member of the gelsolin/villin superfamily (Silacci et al., (2004) Cell Mol Life Sci, 61, 2614-23), located intracellularly (cytosol, mitochondria) and extracellularly (blood, plasma) (Koya et al., (2000) J Biol Chem, 275, 15343-49). It is a key regulator of actin filament assembly and disassembly and is involved in maintaining cell structure and motility (Silacci et al., (2004) Cell Mol Life Sci, 61, 2614-23). Increased GSN expression is associated with interstitial fibrosis and inflammation (Oikonomou et al., (2009) Thorax, 64, 467-75), likely due to the GSN-mediated destabilization of cytoskeleton and increased movement of platelets and immune infiltrate, and GSN^{-/-} mice are shown to develop decreased pulmonary fibrosis and inflammation (Oikonomou et al., (2009) Thorax, 64, 467-75). In the heart, GSN catalyzes the disassembly and degradation of myocardial proteins (Yang et al., (2000) Circulation, 102, 3046-52), and its increased expression is detected in failing human hearts (Yang et al., (2000) Circulation, 102, 3046-52). It has been suggested that GSN interacts with hypoxia inducible factor 1 (HIF1A), a master transcriptional regulator of the cellular and systemic responses to hypoxia, that is known to play an essential role in the pathophysiology of ischemic cardiovascular disease (Richard et al., (2003) Circulation, 107, 2227-32).

MYL2 (myosin regulatory light chain 2) is a cardiac-specific protein. MYL2 dimerizes with cardiac myosin beta (or slow) heavy chain, and its phosphorylation by Ca⁺ triggers cardiac contractions. Mutations in MYL2 or abnormalities in MYL2 expression are associated with cardiomyopathy (Richard et al., (2003) Circulation, 107, 2227-32), heart failure (Poetter et al., (1996) Nat Genet, 13, 63-69), and left ventricular hypertrophy and familial hypertrophy (Flavigny et al., (1998) J Mol Med, 76, 208-14; Kabaeva et al., (2002) Eur J Hum Genet, 10, 741-48). Expression of MYL2 is altered in chagasic hearts (Cunha-Neto et al., (2005) Am J Pathol, 167, 305-13) and isolated cardiomyocytes infected by T. cruzi (Goldenberg et al., (2009) Microbes Infect, 11, 1140-49), and it is suggested that T. cruzi-induced immunoglobulin G autoantibodies and delayed type hypersensitivity to cardiac myosin contribute to disease pathogenesis (Leon and Engman, (2001) Int J Parasitol, 31, 555-61; Leon et al., (2004) Infect Immun, 72, 3410-17). Certain aspects of the described studies provide the first evidence that the plasma release of MYL2 is linked to disease severity in chagasic patients and indicative of the extent of cardiac muscle injury during Chagas disease development.

VIM is a member of the intermediate filament network, and it is primarily expressed by mesenchymal cells and found in connective tissue. Along with microtubules and actin microfilaments, VIM plays an important role in maintaining cell shape, integrity of the cytoplasm, and stabilizing cytoskeletal interactions (Katsumoto et al., (1990) Biol Cell, 68, 139-46). Vimentin is also shown to be localized in the carotid artery and heart valves and serves as a target antigen of peripheral and heart-infiltrating T cells during valvular disease (Fae et al., (2008) J Autoimmun, 31, 136-41). Increased detection of vimentin in the heart is indicative of a fibrotic process, as infiltrating fibroblasts replace damaged cardiomyocytes in disease conditions and has been identified by proteomic inventory of myocardial proteins in patients with Chagas disease (Teixeira et al., (2006) Braz J Med Biol Res, 39, 1549-62). Results obtained through IPA analysis indicated that VIM modulates NOS2 and is indirectly linked to IL-1β and TNF-α expression in the disease state.

It is demonstrated herein that depletion of high-abundance plasma proteins enhanced the protein discovery of low-abundance proteins by 2D-GE. Pathological events, i.e., persistent inflammation and oxidative stress, associated with Chagas heart disease, and the beneficial effects of antioxidant and anti-parasite therapies in preserving the cardiac function, were reflected in the plasma protein profile of experimentally infected rodents. These proteomic studies provide the first indication that lipid/fatty acid metabolism is dysregulated and of pathologic significance in Chagas disease. Importantly, protein biomarkers (GSN, MYL2, VIM, MYH11, VCL, and PLG) were identified that have utility in diagnosing the presence or severity of Chagas disease, and/or identifying the patients at risk of developing clinical symptoms of Chagas disease.

Certain embodiments are directed to methods of detecting Chagas disease in a biological sample, comprising the step of measuring the presence of at least one protein selected from the group consisting of GSN, MYL2, VIM, MYH11, VCL, and PLG in said sample, wherein elevated levels of GSN, MYL2, VIM, MYH11, VCL, and PLG is indicative of Chagas disease in the subject from which the sample was obtained. Generally, the biological sample is a diagnostic sample from a human or non-human animal. Representative samples include but are not limited to a tissue sample, a plasma sample, or a blood sample. Generally, GSN, MYL2, VIM, MYH11, VCL, and PLG may be detected by any assay known to one of ordinary skill in this art. Representative assays include but are not limited to a Western blot assay, an ELISA assay, an immunofluorescence assay, an immunoprecipitation assay, and a radioimmunoassay. Preferably, the assay determines the concentration of GSN, MYL2, VIM, MYH11, VCL, or PLG in said sample to be ≧50% greater than normal controls. Normal controls are mammals not having cardiac disease or are not at risk of developing heart failure due to chagasic or other etiologies.

In yet another embodiment of the present invention, there is provided a serodiagnostic kit for determining the presence and/or severity of Chagas disease, said kit comprising: (a) the antibody directed against GSN, MYL2, VIM, MYH11, VCL, and/or PLG, wherein the antibody is linked to a reporter molecule; (b) a buffer; and, (c) a reagent for detection of the reporter molecule. Useful antibodies directed against GSN, MYL2, VIM, MYH11, VCL, and PLG are well known to those with ordinary skill in this art. Representative reporter molecules include but are not limited to luciferase, horseradish peroxidase, P-galactosidase, and fluorescent labels.

The following examples as well as the figures are included to demonstrate embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Animals and parasites. Sprague Dawley rats (4-5-weeks-old, Harlan Labs) were infected with T. cruzi (SylvioX10/4, 50,000 trypomastigotes/rat, intraperitoneal). Rats were given 1.3 mM PBN (beginning day 0, throughout the course of infection) and/or 0.7 mM BZ (beginning day 40 pi, for three weeks) in drinking water. The plasma samples were obtained at acute (27-45 days post-infection (dpi)) and chronic (>150 dpi) stages of infection and disease development.

Rat plasma collection and removal of high-abundance proteins. Whole blood was collected in K₃EDTA vacutainers and kept on ice for 30 min. The samples were centrifuged for 15 min at 3,000 g, and the lipid-containing upper layer was removed. The abundant proteins (e.g., albumin, α1-acid glycoprotein, α1-antitrypsin, α2-5 macroglobulin, apolipoproteins (ApoA-I, ApoA-II), fibrinogen, haptoglobin, immunoglobulins (IgA, IgG, and IgM), and transferrin) in plasma were removed using ProteomeLab IgY-12 LC 10 affinity spin columns (Beckman Coulter). The plasma samples depleted of abundant proteins were then concentrated by using Amicon® ultra centrifugal filters (3 kDa cut-off, from Millipore) and desalted by PD-10 desalting columns (GE Healthcare). Protein content was determined by using the Bradford assay (Bio-Rad).

Two-dimensional gel electrophoresis. The 11-cm immobilized pH gradient (IPG) strips (pH: 3-10 or 5-8, Bio-Rad) were rehydrated at 50 V for 12 h with 250-μl rehydration buffer (1 M thiourea, 8 M urea, 2% CHAPS, 1% dithiothreitol, and 0.2% ampholytes) containing 200-μg protein samples and 0.002% of bromophenol blue. Isoelectric focusing was performed at 500 V for 1 h, 1000 V for 1 h, 8000 V for 2 h, and then at 8000 V for a total of 50,000 Vh. The IPG strips were suspended in equilibration buffer (50 mM Tris-HCI, pH 6.8, 6 M urea, 20% glycerol) and sequentially incubated for 15 min each in presence of 2% DTT/2% SDS (reducing conditions) and 2.5% iodoacetamide/2% SDS (alkylating conditions). Equilibrated IPG strips were subjected to second-dimension electrophoresis by using 8-10% linear gradient precast Tris-HCI gels (BioRad) on a PROTEAN plus Dodeca Cell System at 75 V for 1 h and then at 120 V until the dye front reached the bottom of the gel. Gels were fixed in 10% methanol/7% acetic acid, stained with SYPRO Ruby (BioRad), destained in 10% ethanol/7% acetic acid, and imaged by using a high-resolution ProXPRESS Proteomic Imaging System (Perkin Elmer).

Image analysis. In total, 28 SYPRO Ruby-stained 2D gels (n=4/group) were digitalized on a ProXPRESS Proteomic Imaging System (Perkin Elmer), and the images were analyzed on Progenesis SameSpots™ software 2.0 (NonLinear Dynamics). Normalized spot volumes, i.e., the volume of each spot over the volume of all spots in the gel, were used for comparison of the different groups, and candidates were identified as protein spots that changed at least 2-fold versus their specific control. Statistical significance was assessed by a two-tailed Student's t-test and analysis of variance test (ANOVA), and p values of <0.05 were considered significant for comparison between control and experimental data.

Detection of carbonyl proteins. IPG strips were loaded with protein samples and subjected to 1^{st}-dimension isoelectric focusing, as above. IPG strips were then incubated with 1 ml of 3% SDS and 5 mM 2,4-dinitrophenylhydrazine (DNPH)/10% trifluoroacetic acid for 25 min. After neutralization with 1.5 ml of 2 M Tris, 30% glycerol, DNP-derivatized protein samples in IPG strips were subjected to second-dimension electrophoresis by using 8-10% gradient precast gels (BioRad), and transferred to PVDF membranes. Membranes were hybridized with rabbit anti-DNP antibody (1:300 dilution, Invitrogen) and HRP-conjugated goat anti-rabbit secondary antibody (1:4000 dilution, Sigma), and signal detected by using an Amersham™ ECL Plus system, according to the manufacturer's protocol (Levine et al., (1990) Methods Enzymol, 186, 464-78; Wen and Garg, (2004) Free Radic Biol Med, 37, 2072-81). Images were visualized, digitized, and signal was quantified by densitometry by using a FluorChem 8800 image analyzer (Alpha Innotech).

Mass spectrometry and protein identification. Selected spots (1-mm) were excised from gels and submitted to trypsin proteolysis as described (Dhiman et al., (2008) Am J Pathol, 173, 728-40). In brief, gel spots were incubated at 37° C. for 30 min in 50 mM NH₄HCO₃, dehydrated twice for 5 min each in 100-μl acetonitrile, dried, and in-gel proteins were digested at 37° C. for 6 h with 10 μL of trypsin solution (1% trypsin in 25 mM ammonium bicarbonate). After digestion, 1 μl of peptide mixture was directly spotted onto a MALDI TOF MS/MS target plate with 1 μl of alpha-cyano-4-hydroxycinnamic acid matrix solution (5 mg/ml in 50% acetonitrile). Peptides were analyzed by using a MALDI TOF/TOF ABI 4800 Proteomics Analyzer (Applied Biosystems). The Applied Biosystems software package included the 4000 Series Explorer (v. 3.6 RC1) with Oracle Database Schema Version (v. 3.19.0) and Data Version (3.80.0) to acquire and analyze MS and MS/MS spectral data. The instrument was operated in a positive ion reflectron mode with the focus mass set at 1700 Da (mass range: 850-3000 Da). For MS data, 1000-2000 laser shots were acquired and averaged from each protein spot. Automatic external calibration was performed by using a peptide mixture with the reference masses 904.468, 1296.685, 1570.677, and 2465.199. MALDI MS/MS was performed on several (Bastos et al., (2010) PLoS Negl Trop Dis, 4, e711; Urbina, (2010) Acta Trop, 115, 55-68; Maya et al., (2010) Biol Res, 43, 323-31; Marin-Neto et al., (2007) Circulation, 115, 1109-23; Saravia et al., (2011) Arch Pathol Lab Med, 135, 243-48; Rassi et al., (2006) N Engl J Med, 355, 799-808) abundant ions from each protein spot. A 1 kV positive ion MS/MS method was used to acquire data under post-source decay (PSD) conditions. The instrument precursor selection window was +/−3 Da. Automatic external calibration was performed by using reference fragment masses 175.120, 480.257, 684.347, 1056.475, and 1441.635 (from precursor mass 1570.700).

Applied Biosystems GPS Explorer™ (v.3.6) software was used in conjunction with MASCOT to search the respective protein database by using both MS and MS/MS spectral data for protein identification. Protein match probabilities were determined by using expectation values and/or MASCOT protein scores. The MS peak filtering included the following parameters: a mass range of 800 Da to 3000 Da; minimum S/N filter=10; mass exclusion list tolerance=0.5 Da; and mass exclusion list for trypsin and keratin-containing compounds included masses 842.51, 870.45, 1045.56, 1179.60, 1277.71, 1475.79, and 2211.1. The MS/MS peak filtering included the following parameters: minimum S/N filter=10, maximum missed cleavages=1, fixed modification of carbamidomethyl (C), variable modifications due to oxidation (M), precursor tolerance=0.2 Da, MS/MS fragment tolerance=0.3 Da, mass=monoisotopic, and peptide charges=+1. The significance of a protein match, based on the peptide mass fingerprint (PMF) in the MS and the MS/MS data from several precursor ions, is presented as expectation values (p<0.001).

The online tool FindMod (accessible on the World Wide Web at expasy.org/tools/findmod.html) was used to predict post-translational modifications on unassigned peaks from MALDI-TOF/TOF MS analysis. This tool calculates the differences between experimentally determined peptide masses and theoretical masses calculated from a specific protein sequence and allocate them to 22 types of post-translational modifications existent in the UniProt knowledge base (accessible on the World Wide Web at uniprot.org/). Tandem mass spectra were searched against the UniProt rat proteome database (accessible on the World Wide Web at expasy.org; 02/2011 release).

Functional analysis. All datasets were assessed by using Ingenuity Pathways Analysis (Ingenuity Systems®) and GOA-UniProt (released on Feb. 20, 2011) to recognize the function of the identified proteins. These two software programs retrieve a set of biological information such as gene name, sub-cellular location, tissue specificity, function, and association with disease; and then integrate the identified proteins into networks and signaling pathways with biological meaning and significance. A list of all gene ontology (GO) terms was obtained, and the frequency of annotation and information content of each term were calculated as described (Yu et al., (2003) Proteomics, 3, 2240-48). Briefly, an e-value was calculated by estimating the probability of a random set of proteins having a frequency of annotation for that term greater than the frequency obtained in the real set, and a threshold of 10⁻³ was set to retrieve significant molecular functions and biological processes. With these parameters, the most informative and significantly over-represented GO terms in the dataset were highlighted.

Human plasma samples. The criteria for characterization of seropositive chagasic subjects exhibiting variable degrees of cardiac disease (CD0-CD3), seronegative cardiomyopathy patients of other etiologies, and seronegative, healthy individuals exhibiting no history or clinical symptoms of cardiac disease were described (Wen et al., (2006) Free Rad Biol Med, 41, 270-76). Human sera samples were collected.

Western blotting. A 20-μg aliquot of each protein sample was resolved on 10% acrylamide gels and transferred to PVDF membranes by using a wet, vertical Criterion Blotter (Bio-Rad). Membranes were blocked for 1 h with 5% nonfat dry milk (NFDM, Bio-Rad) in 50 mM Tris-HCI (pH 7.4) containing 150 mM NaCI and 0.05% Tween-20 (TBST). All antibody dilutions were made in 5% NFDM-TBST. Membranes were incubated overnight at 4° C. with antibodies against GSN, MYL2, and VIM (1:2000 dilution, Santa Cruz). Membranes were washed thrice, incubated with HRP-conjugated secondary antibody (1:4000, Sigma) for 1 h, and signal was developed using the Amersham™ ECL Plus system. Membranes were stained with Coomassie blue G250 (Bio-Rad) to confirm an equal loading of samples. Images were visualized and digitized, and densitometric analysis was performed by using a FluorChem 8800 system.

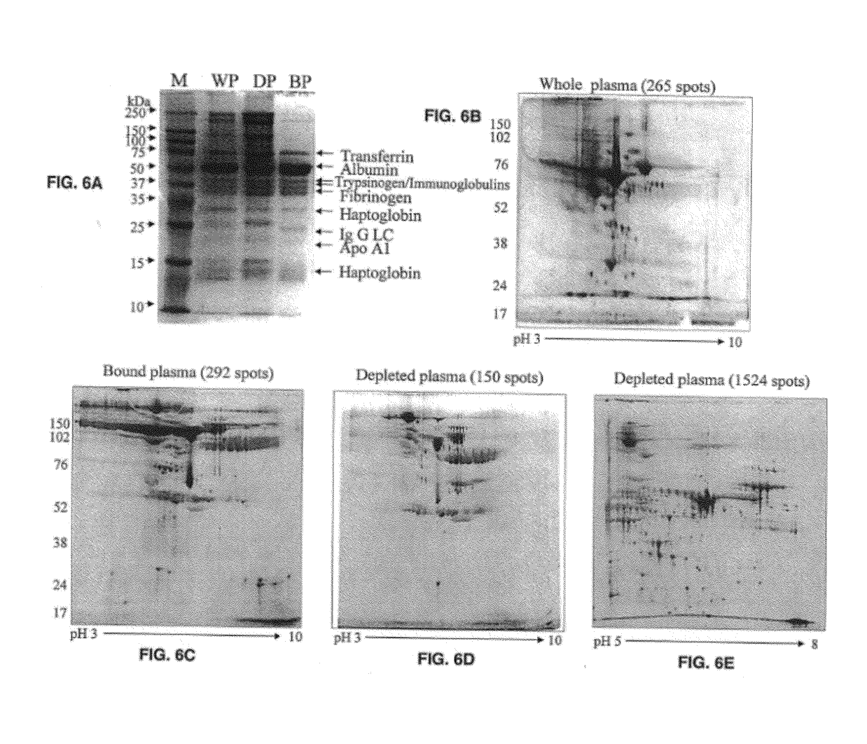

Serum depletion of high-abundance proteins enhanced the detection of low-abundance proteins. Plasma is the most clinically abundant specimen; however, because of a dynamic range of protein concentration, the high-abundance proteins interfere with the detection of low-abundance potential biomarker proteins. To enhance one's ability to detect low abundance proteins, IgY-12 LC10 immuno-affinity columns were employed that provided a >90% depletion of 12 major proteins from the plasma samples (FIG. 6A). To verify whether depletion of abundant proteins enhanced detection of low-abundance proteins, 2D-GE was performed, and the gel images acquired after staining with SYPRO Ruby (FIG. 6B-6D) were analyzed. When using pH 3-10 IPG strips, whole plasma and depleted plasma proteins resolved on the acidic side or in the middle portion of the 2^{nd}-dimension 8-16% gradient gel (FIG. 6B-6D). The narrow-range IPG strips (pH 5-8) provided an enhanced resolution of depleted plasma proteins and the detection of 5-fold more spots than were detected in whole plasma (1524 versus 265, FIGS. 6B and 6E). Therefore, pH 5-8 IPG strips and depleted plasma were used for all additional studies.

2D-GE analysis of changes in plasma proteome by T. cruzi infection. Sprague Dawley rats were infected with T. cruzi and treated with phenyl-α-tert-butyl nitrone (PBN, antioxidant) with or without benznidazole (BZ, anti-parasite drug). Plasma samples were collected at day 25 (acute stage) and 150 (chronic stage) post-infection, and depleted of high-abundance proteins to enrich detection of low-abundance proteins by 2D-gel electrophoresis. Gel images of plasma samples from normal rats (RN), rats that were acutely infected (RA) and treated with PBN (RAP), and chronically infected rats (RC) that were treated with PBN (RCP), BZ (RCB), or PBN and BZ (RCPB) were analyzed on Progenesis SameSpotst™ software and normalized spot volumes were used for comparison of different groups. Proteins spots with >2-fold change in chagasic plasma were subjected to MALDI-TOF MS/MS analysis. The significance of a protein match, based on the peptide mass fingerprint in the MS and the MS/MS data, is presented as expectation values (p<0.001). The putative biological function and cellular location were identified using Ingenuity Pathway Analysis and UniProt software.

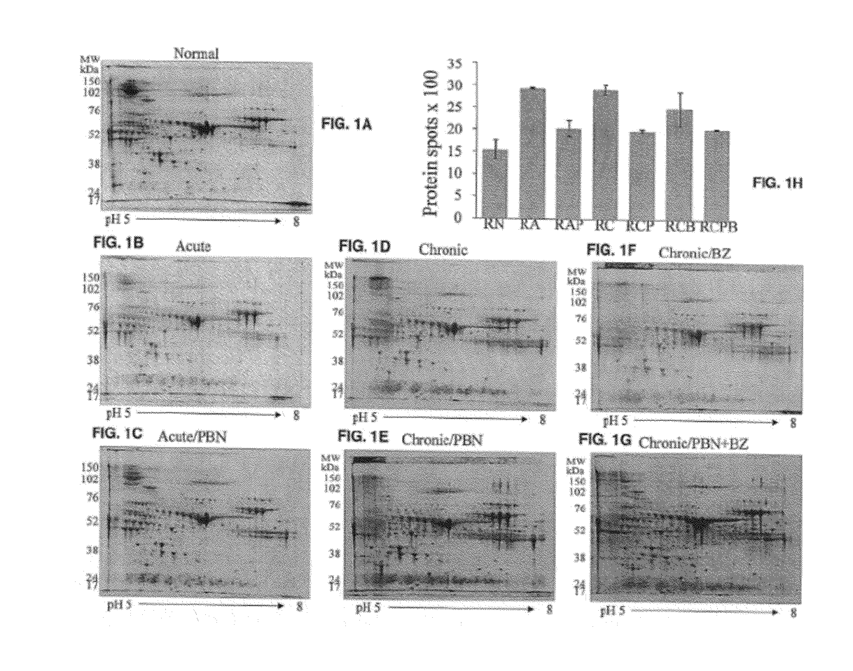

The depleted plasma proteome of normal rats (RN), acutely infected (RA) treated with PBN (RAP), and chronically infected rats (RC) treated with PBN (RCP), BZ (RCB), or PBN and BZ (RCPB) were analyzed. Plasma samples were resolved to obtain eight gel replicas per group. Representative images are shown in FIGS. 1A-1G. Gel images were aligned by using Progenesis SameSpots™ and protein spots identified. In total, 1524, 2915, 2015, 2900, 1979, 2486, and 2026 protein spots were observed in the RN, RA, RAP, RC, RCP, RCB, and RCPB groups, respectively (FIG. 1H). The maximal increase in the number of protein spots was associated with acute infection (RA) or chronic disease (RC) status. PBN treatment resulted in, respectively, 65% and 63.6-66.9% normalization of plasma protein profile of acutely (RAP) and chronically (RCP or RCPB) infected rats. BZ treatment resulted in a moderate (30.1%) decline in the plasma proteome profile of chronically infected rats (FIG. 1H).

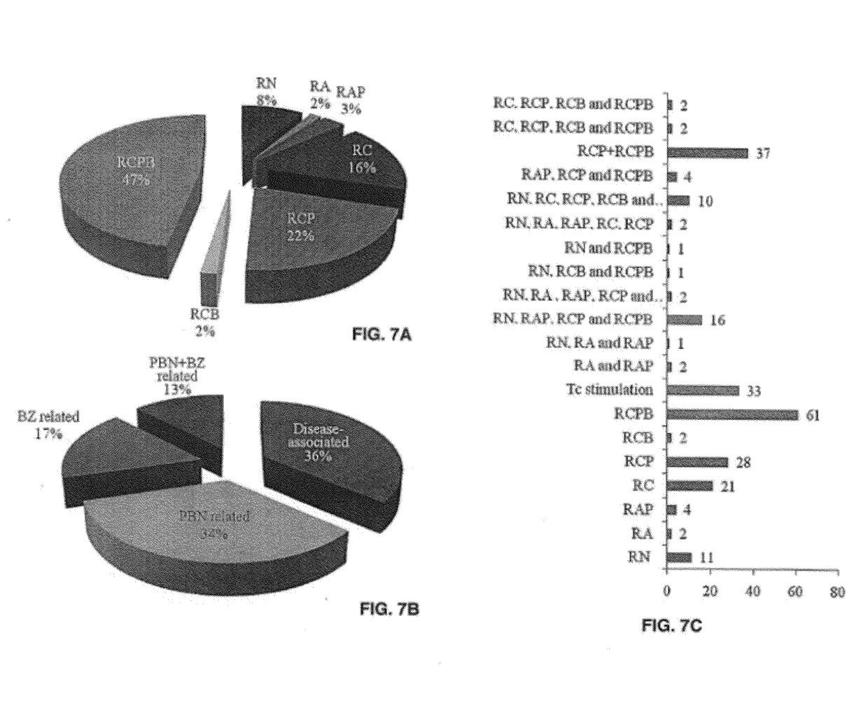

Densitometric analysis of the protein spot signal by Progenesis SameSpots™ identified ˜1000 spots that were altered in expression by ≧2-fold (p_{ANOVA}<0.05). Of these, 325 protein spots were reproducibly identified to be differentially expressed in response to T. cruzi infection and/or treatment in at least three different experiments. The analysis suggested that ˜8% of the differentially expressed protein spots were down regulated in response to infection, and 92% were enhanced in expression in infected rats (with or without treatment) when compared to those in normal controls (FIG. 7A). 34%, 17%, and 13% of the differentially expressed proteins were identified in chagasic plasma of rats treated with PBN, BZ, and PBN/BZ, respectively (FIG. 7B, Table 1a). Some proteins were strictly stimulated in response to T. cruzi (33 spots), and others were strictly associated with acute infection (2 spots) or chronic infection (2 spots) (FIG. 7C). 41 protein spots that were up regulated by PBN treatment in acute (4 spots) and chronic plasma (37 spots) as compared to the spots from normal controls (FIG. 7C) were also identified. Results are shown in Table 1b and together these results indicated that during Chagas disease, oxidative stress and parasite persistence contribute to alterations in the plasma proteome profile.

TABLE la
List of Genes/Proteins Differentially Expressed in Response to T. Cruzi Infection

| | | | | Putative | |
|---|---|---|---|---|---|
| | | | | Biological | Putative Cellul [symbol] |
| Spot# | Gene Name | Protein Name | Accession No. | Function | Location |
| 12, 180 | KRT1 | Keratin, type II | gi/120474989 | Oxidative stress res [symbol] | Membrane |
| 34 | TTC37 | KIAA0372 gene product | gi/149058911 | Protein binding | N/A |
| 37, 593 | SRPRB | Bal-667 | gi/33086638 | Iron homeostasis | Extracellular |
| 49, 673 | IGH-1 | Igh-la protein | gi/299352 | Antigen binding | Extracellular |
| 52, 54, 302 | TF | Transferrin | gi/1854476 | Transport | Extracellular |
| 53, 225 | SroTP | Serotransferrin | gi/61556986 | Proteolysis | Membrane |
| 72 | IDH3A | Isocitrate dehydrogenase 3a | gi/149041700 | Metabolism | Mitochondria |
| 73, 705, 787 | ALB | Alpha-1-inhibitor 3 | gi/83816939 | Inflammatory respo [symbol] | Extracellular |
| 77 | MUG1 | Murinoglobulin-1 | gi/12831225 | Acute-phase respo [symbol] | Extracellular |
| 88 | IGHG- | g-2a immunoglobulin heavy c [symbol] | gi/1220486 | Antigen binding | N/A |
| 94 | FGB | Fibrinogen beta chain | gi/124106312 | Signal transductio [symbol] | Membrane |
| 106 | CDK5RAP2 | CDK5 regulatory associate [symbol] | gi/109476582 | tRNA modificatio [symbol] | Cytoplasm |
| | | 2 | | | |
| 115, 123 | SERPINAL1 | Alpha-1-antiproteinase | gi/112889 | Acute phase respo [symbol] | Extracellular |
| 149 | PUS1 | Pseudouridine synthase 1 | gi/149063707 | tRNA processing | Mitochondria |
| 153 | MYL2 | Myosin, light polypeptide 2 | gi/149067749 | Muscle contractio [symbol] | Cytosol |
| 177 | AIM | Alpha-1-macroglobulin precu [symbol] | gi/21955142 | Protein binding | Extracellular |
| 181 | SERPINA3L | Serine protease inhibitor A3L | gi/2507387 | Inhibitory protein | Extracellular |
| 184, 257, 328 | CFH | Complement inhibitory factor | gi/15485713 | Immune response | Cytoplasm |
| 207, 555 | CCHL1A1 | Calcium channel alpha-1 subu [symbol] | gi/1184038 | Transport | Membrane |
| 215 | GPT | Glutamic pyruvic transami [symbol] | gi/149066073 | Gluconeogenesis | Cytoplasm |
| 266 | CEP350 | Centrosome-associated protei [symbol] | gi/18027304 | Cytoskeleton | Cytoplasm |
| 299 | UMPS | Uridine monophosphate sy [symbol] | gi/149060638 | Metabolism | Cytoplasm |
| 315 | ITIH4 | Inter-alpha-inhibitor H4 he [symbol] | gi/126722991 | Metabolism | Extracellular |
| 334 | ZNF689 | Zinc finger, HIT type 6 | gi/157818873 | Transcription | Nucleus |
| 341 | Nmag 2782 | Na7Ca⁺ antiporter | gi/8825638 | Transport | Membrane |
| 355, 359 | PLG | Plasminogen | gi/16758216 | Tissue remodeling | Extracellular |
| 382, 385, 378, | CFB | Complement factor B | gill49027999 | Immune response | Extracellular |
| 572, 578, 595 | | | | | |
| 389 | GSN | Gelsolin | gi/149038928 | Cytoskeleton | Cytosol |
| 430 | SCD2 | Stearyl-CoA desaturase 2 | gi/1763027 | Fatty acid synthes [symbol] | Membrane |
| 434, 678 | COG1 | Component of golgi compl [symbol] | gi/149054700 | Transport | Membrane |
| 439 | C4BPA | C4b-binding protein alpha | gi/2493792 | Innate immunity | Extracellular |
| 452 | AFM | Afamin, Albumin-binding | gi/60688254 | Transport | Extracellular |
| 464 | IGH-6 | Immunoglobulin heavy cha [symbol] | gi/62201965 | Antigen binding | N/A |
| 479 | IGHM | Ig mu chain C region | gi/111977 | Immune response | Membrane |
| 493 | ZFP637 | Zinc finger protein 637 | gi/201027426 | Protein binding | Intracellular |
| 515 | CESIC/ES2 | Carboxylesterase | gi/468766 | Metabolism | Endoplasmic re [symbol] |
| 524 | ERCC4 | DNA repair endonuclease [symbol] | gi/109487684 | DNA repair | Nucleus |
| 551, 587, 908 | C3 | Complement C3 | gi/158138561 | Inflammation | Extracellular |
| 559 | HPX | Hemopexin | gi/122065203 | Heme scavenging | Extracellular |
| 571 | GC | Group specific component | gi/51260133 | Vitamin D binding | Extracellular |
| 576, 620 | Act-248 | SERPIN-like protein | gi/32527753 | Inflammation resp [symbol] | Extracellular |
| 586, 614 | SERPINC1 | Serine/cysteine peptidase in [symbol] | gi/58865630 | Blood clotting | Extracellular |
| 592 | DNASE1L1 | Deoxyribonuclease 1-like | g/149029874 | DNA catabolism | Endoplasmic re [symbol] |
| 604 | APOH | Beta-2-glycoprotein 1 | gi/57528174 | Transport | Cell surface |
| 606 | GC | Vitamin D binding protein | gi/203927 | Transport | Extracellular |
| 627 | MAPI | LMW T-kininogen I | gi/205085 | Acute-phase respo [symbol] | Extracellular |
| 638 | LOC501738 | Immune activating receptor | gi/264681503 | Receptor activity | N/A |
| 649 | SYNE2 | Nesprin-2 like | gi/109478368 | Cytoskeleton | Nuclear |
| 651 | DSTN | Destrin | gi/75991707 | Actin binding | Cytoplasm |
| 655 | DLGAP2 | Disks large-associated prote [symbol] | gi/16758774 | Synapse transmiss [symbol] | Membrane |
| 672 | SERPINF | Serine/cysteine peptidase in [symbol] | gi/29293811 | Inhibitory protein | Extracellular |
| 676 | rCG33447 | Hypothetical | gi/149052919 | N/A | N/A |
| 691 | APOA4 | Apolipoprotein A-IV | gi/114008 | Lipid binding | Extracellular |
| 860 | KRT10 | Keratin, type I | gi/57012436 | Protein binding | Intermediate fi [symbol] |
| 966 | Hypothetical | CMRF-35-like molecule-7 | gi/109511428 | Immunity | Membrane |
| 897 | MY05A | Myosin Va | gi/149019170 | Myosin complex | Cytoplasm |
| 715 | SERPINA1 | Proteinase inhibitor-like | gi/930263 | Protein binding | Extracellular |
| 724 | ALB | Serum albumin | gi/158138568 | Transport | Extracellular |
| 728 | SYMPK | Symplekin-C | gi/62531221 | Protein binding | Nucleus |
| 733 | DECR2 | 2,4-dienoyl-CoA reductase | gi/25282441 | Metabolism | Peroxisome |
| 740 | PRPH | Peripherin | gi/166063971 | Cytoskeleton | Intermediate fil [symbol] |
| 745 | PFKFB1 | 6-phosphofructo-2-kinase 1 | gi/77020248 | Metabolism | Cytoplasm |
| 749 | MY05B | Myosin-Vb | gi/8393817 | Myosin complex | Intracellular |
| 766 | SCG2 | Secretogranin 2 | gi/149016236 | Inflammation | Extracellular |
| 779, 909 | VIM | Vimentin | gi/149021116 | Cell integrity | Cytoplasm |
| 796 | Hypothetical p [symbol] | | gi/109468251 | N/A | N/A |
| 814 | RBM15B | RNA binding motif 15B | gi/109483938 | RNA splicing | Nucleoplasm |
| 817 | BRPF1 | Bromodomain/PHD finger- | gi/109472470 | Transcription | Cytoplasm |
| | | protein 1 | | | |
| 820, 1081 | BFAR | Bifunctional apoptosis regu [symbol] | gi/61557021 | Apoptosis | Membrane |
| 827 | GNAT | Guanine binding protein alp [symbol] | gi/84662745 | N/A | Cytoplasm |
| 832 | PSMB | Proteasome beta-type subu [symbol] | gi/9653292 | Protein catabolism | Cytoplasm |
| 867 | RAI14 | Ankycorbin | gi/58865464 | N/A | Cytoplasm |
| 881 | CHD4 | Chromodomain helicase D [symbol] | gi/149049419 | Transcription | Nucleus |
| | | protein 4 | | | |
| 900 | rCG25416 | Transferrin region | gi/149018747 | N/A | N/A |
| 906 | APOE | Apolipoprotein E | gi/149056721 | Oxidative stress re [symbol] | Chylomicron |
| 979 | rCG25357 | | gi/149018900 | N/A | N/A |
| 992 | APOA1 | Apolipoprotein A-I | gi/2145143 | Transport | Membrane |
| 1019 | ZNHIT6 | Hypothetical protein FLJ20 | gi/149026162 | Protein binding | Pre-snoRNP co [symbol] |

[symbol]

indicates data missing or illegible when filed

TABLE 1b
Differential Expression in Response to T. Cruzi Infection

| | | | e-value | | | | | | | |
|---|---|---|---|---|---|---|---|---|---|---|
| Spot# | MW (Da) | pI | p < 0.001 | RN* | RA* | RAP* | RC* | RCP* | RCB* | RCPB* |
| 12, 180 | 65059.2 | 8.04 | 9.102E−09 | 4.70 | 4.03 | 21.47 | 9.92 | 7.22 | 9.47 | 11.17 |
| 34 | 68849.3 | 6.22 | 0.001 | 2.29 | 0.64 | 0.37 | 0.31 | 0.45 | 0.37 | 0.25 |
| 37, 593 | 109545.9 | 8.35 | 5.319E−08 | 2.14 | 5.33 | 14.38 | 6.46 | 6.48 | 3.21 | 8.61 |
| 49, 673 | 52500 | 7.23 | 0.132 | 7.89 | 9.90 | 19.12 | 50.91 | 13.39 | 3.98 | 4.48 |
| 52, 54, 302 | 78538.3 | 6.94 | 5.55E−08 | 9.83 | 5.80 | 9.80 | 2.85 | 14.45 | 0.60 | 8.06 |
| 53, 225 | 78512.5 | 7.14 | 1.671E−07 | 6.78 | 6.20 | 8.76 | 2.05 | 15.12 | 1.72 | 5.60 |
| 72 | 30334.3 | 5.83 | 6.694E−07 | 39.31 | 10.26 | 18.10 | 76.03 | 16.43 | 83.09 | 13.00 |
| 73, 705, 787 | 165038.2 | 5.7 | 6.561E−09 | 86.41 | 19.56 | 46.94 | 136.2 | 38.67 | 216.9 | 28.67 |
| 77 | 166589.9 | 5.68 | 0.00007856 | 28.44 | 16.28 | 16.3 | 47.41 | 8.675 | 107.7 | 20.1 |
| 88 | 52242.6 | 8.15 | 1.24E−08 | 2.54 | 1.45 | 74.61 | 38.51 | 40.49 | 4.75 | 17.20 |
| 94 | 54827.9 | 7.9 | 0.0008907 | 0.33 | 0.51 | 1.84 | 1.87 | 2.55 | 0.55 | 0.53 |
| 106 | 208168.7 | 5.27 | 0.001 | 1.47 | 0.85 | 1.70 | 2.22 | 6.94 | 2.21 | 1.67 |
| 115, 123 | 46277.6 | 5.7 | 0.272 | 11.10 | 6.35 | 22.27 | 10.54 | 10.96 | 8.36 | 18.61 |
| 149 | 44463.4 | 8.1 | 0.000911 | 6.34 | 1.67 | 0.78 | 6.50 | 4.39 | 1.16 | 1.17 |
| 153 | 17669.7 | 4.96 | 0.00004491 | 9.21 | 3.03 | 27.07 | 38.86 | 10.34 | 2.37 | 2.66 |
| 177 | 168421.9 | 6.46 | 2.312E−10 | 2.88 | 1.84 | 3.71 | 28.06 | 1.60 | 2.77 | 2.87 |
| 181 | 46419.1 | 5.48 | 0.007 | 1.83 | 3.57 | 3.99 | 17.08 | 1.90 | 7.53 | 3.22 |
| 184, 257, 328 | 144813.9 | 6.52 | 0.001 | 3.69 | 9.51 | 4.54 | 2.46 | 5.85 | 6.15 | 3.32 |
| 207, 555 | 13893.9 | 6.52 | 0.0001206 | 15.79 | 10.78 | 13.23 | 13.97 | 9.12 | 11.32 | 18.65 |
| 215 | 50475.5 | 6.63 | 2.201E−08 | 3.47 | 1.20 | 1.73 | 26.17 | 1.97 | 3.23 | 1.98 |
| 266 | 166809.6 | 8.63 | 0.012 | 0.56 | 0.22 | 0.75 | 0.56 | 0.54 | 1.43 | 1.22 |
| 299 | 33345.2 | 6.02 | 0.00001753 | 2.05 | 1.45 | 1.92 | 0.66 | 1.17 | 1.17 | 2.01 |
| 315 | 103861.8 | 5.82 | 0.00001561 | 1.93 | 2.95 | 6.24 | 7.00 | 8.23 | 5.19 | 3.92 |
| 334 | 53374.8 | 5.51 | 4.73E−08 | 26.07 | 0.59 | 0.61 | 1.00 | 0.71 | 1.05 | 0.45 |
| 341 | 11217.6 | 8.91 | 5.217E−06 | 1.87 | 1.50 | 0.59 | 1.10 | 1.10 | 1.44 | 6.59 |
| 355, 359 | 93213.9 | 6.79 | 0.00001998 | 3.15 | 4.34 | 2.99 | 3.15 | 2.20 | 3.29 | 3.45 |
| 382, 385, 378, | 83735.6 | 6.05 | 1.675E−10 | 5.91 | 43.78 | 33.76 | 30.02 | 3.29 | 23.45 | 5.94 |
| 572, 578, 595 | | | | | | | | | | |
| 389 | 86314.2 | 5.76 | 6.661E−14 | 6.51 | 70.05 | 37.55 | 38.06 | 4.32 | 44.17 | 8.39 |
| 430 | 3479.7 | 9.9 | 2.661E−09 | 11.28 | 40.97 | 22.40 | 9.29 | 8.21 | 13.65 | 11.38 |
| 434, 678 | 72098.8 | 8.06 | 1.465E−08 | 29.10 | 110.5 | 55.14 | 34.52 | 24.63 | 43.20 | 30.09 |
| 439 | 64277.8 | 7.06 | 9.889E−09 | 4.21 | 1.72 | 4.04 | 13.64 | 14.99 | 1.36 | 7.56 |
| 452 | 54205.3 | 5.8 | 5.923E−09 | 25.61 | 13.68 | 18.92 | 17.57 | 10.01 | 59.65 | 55.78 |
| 464 | 69059.5 | 5.69 | 0.00004813 | 5.89 | 7.22 | 3.89 | 3.22 | 6.65 | 4.66 | 7.97 |
| 479 | 38189.1 | 6.72 | 1.038E−11 | 2.72 | 21.09 | 23.96 | 10.08 | 32.56 | 8.65 | 14.62 |
| 493 | 31712.4 | 9.5 | 6.464E−07 | 3.75 | 14.49 | 9.82 | 5.45 | 13.46 | 17.41 | 12.88 |
| 515 | 59196.9 | 5.51 | 5.223E−07 | 17.08 | 3.47 | 6.87 | 8.05 | 7.78 | 24.22 | 19.57 |
| 524 | 110025.1 | 9.2 | 9.067E−09 | 120.8 | 32.57 | 53.74 | 55.47 | 21.96 | 68.54 | 30.55 |
| 551, 587, 908 | 187745.9 | 6.06 | 1.64E−11 | 7.046 | 146.9 | 88.04 | 46.76 | 54.1 | 32.88 | 31.97 |
| 559 | 52059.6 | 7.58 | 0.167 | 26.14 | 16.11 | 24.39 | 38.23 | 27.58 | 17.91 | 22.98 |
| 571 | 55079.6 | 5.65 | 9.36E−12 | 37.07 | 11.96 | 7.84 | 74.44 | 38.63 | 3.75 | 24.61 |
| 576, 620 | 67191 | 6.85 | 1.098E−07 | 10.86 | 14.95 | 18.08 | 16.62 | 16.66 | 2.38 | 11.23 |
| 586, 614 | 52714 | 6.18 | 3.838E−06 | 63.42 | 48.70 | 50.44 | 57.02 | 38.00 | 132.20 | 75.38 |
| 592 | 32437 | 6.31 | 1.664E−07 | 21.16 | 20.04 | 15.61 | 12.37 | 31.55 | 11.70 | 17.68 |
| 604 | 39743.2 | 8.58 | 6.515E−06 | 18.54 | 10.78 | 22.81 | 17.48 | 23.66 | 67.12 | 26.76 |
| 606 | 55089.6 | 5.65 | 0.006 | 15.31 | 5.92 | 9.60 | 9.50 | 5.58 | 11.27 | 10.14 |
| 627 | 48757 | 6.29 | 1.263E−07 | 108.9 | 20.28 | 30.23 | 45.81 | 54.85 | 44.92 | 38.20 |
| 638 | 24023.2 | 9.41 | 0.00001901 | 3.11 | 4.73 | 4.42 | 5.82 | 5.92 | 4.69 | 12.14 |
| 649 | 442636.4 | 5.29 | 1.843E−08 | 52.04 | 10.43 | 40.69 | 33.92 | 37.11 | 26.48 | 25.27 |
| 651 | 18806.7 | 8.19 | 3.884E−10 | 23.61 | 109.0 | 94.03 | 176.80 | 57.85 | 37.12 | 51.53 |
| 655 | 111348.9 | 6.82 | 6.158E−10 | 48.96 | 280.6 | 240.6 | 254.70 | 149.20 | 151.30 | 122.60 |
| 672 | 46493.2 | 6.04 | 0.00003611 | 9.02 | 3.59 | 7.34 | 3.45 | 17.65 | 23.29 | 10.52 |
| 676 | 20157.4 | 9.14 | 0.00001667 | 13.09 | 19.5 | 14.69 | 49.61 | 42.59 | 24.67 | 17.95 |
| 691 | 44428.7 | 5.12 | 1.996E−06 | 46.70 | 42.11 | 49.99 | 39.11 | 16.29 | 65.41 | 14.52 |
| 860 | 56698.6 | 5.1 | 4.594E−08 | 6.32 | 2.94 | 3.06 | 3.63 | 5.79 | 2.64 | 20.82 |
| 966 | 9215.8 | 7.93 | 3.917E−09 | 12.66 | 76.58 | 53.33 | 87.53 | 59.29 | 49.99 | 28.90 |
| 897 | 96579.8 | 9.48 | 5.875E−06 | 24.00 | 9.21 | 4.82 | 16.65 | 31.94 | 11.65 | 19.24 |
| 715 | 22867.8 | 6.06 | 2.851E−07 | 6.35 | 12.36 | 10.83 | 24.11 | 32.20 | 30.96 | 12.93 |
| 724 | 70709.9 | 6.09 | 5.902E−07 | 1.19 | 3.31 | 1.72 | 0.46 | 2.23 | 2.19 | 11.17 |
| 728 | 33433.1 | 4.97 | 1.601E−11 | 0.84 | 1.00 | 0.76 | 0.49 | 1.13 | 2.21 | 7.89 |
| 733 | 31614.4 | 8.51 | 0.0006324 | 1.71 | 1.90 | 2.24 | 2.06 | 2.44 | 1.67 | 7.42 |
| 740 | 54063.5 | 5.32 | 0.0001771 | 14.90 | 5.56 | 7.81 | 4.46 | 7.33 | 4.02 | 13.03 |
| 745 | 55301 | 6.78 | 3.474E−08 | 8.56 | 4.94 | 4.51 | 4.17 | 3.21 | 2.30 | 8.93 |
| 749 | 215240.6 | 6.53 | 6.828E−10 | 5.53 | 9.51 | 4.71 | 2.88 | 1.70 | 2.46 | 19.91 |
| 766 | 61578.7 | 4.69 | 0.000699 | 3.94 | 5.60 | 3.20 | 3.16 | 6.32 | 3.79 | 14.46 |
| 779, 909 | 4131.2 | 9.75 | 2.984E−09 | 1.52 | 6.85 | 5.45 | 36.40 | 6.88 | 3.08 | 7.28 |
| 796 | 53156.7 | 8.32 | 1.702E−07 | 10.25 | 8.42 | 9.97 | 7.48 | 8.33 | 5.11 | 11.64 |
| 814 | 96223.1 | 9.85 | 4.256E−07 | 1.65 | 1.41 | 0.60 | 0.63 | 1.56 | 3.61 | 13.27 |
| 817 | 151416.4 | 8.55 | 0.024 | 10.16 | 21.83 | 12.14 | 8.66 | 29.59 | 25.15 | 31.35 |
| 820, 1081 | 53617.2 | 6.44 | 0.006 | 4.77 | 3.19 | 4.59 | 31.17 | 2.20 | 13.38 | 5.61 |
| 827 | 42416.4 | 5.48 | 4.658E−09 | 11.89 | 15.94 | 17.91 | 3.82 | 17.80 | 10.78 | 12.84 |
| 832 | 15426.5 | 6.82 | 0.014 | 2.47 | 3.16 | 6.23 | 1.86 | 7.55 | 3.47 | 8.94 |
| 867 | 106502 | 5.64 | 0.0002925 | 4.34 | 5.72 | 2.56 | 2.86 | 3.00 | 2.41 | 12.07 |
| 881 | 111042 | 5.43 | 2.167E−10 | 11.78 | 5.61 | 13.92 | 17.58 | 6.68 | 28.89 | 8.62 |
| 900 | 66991.6 | 6.41 | 9.426E−08 | 20.46 | 9.26 | 25.94 | 22.29 | 44.00 | 7.06 | 18.89 |
| 906 | 27354.1 | 7.93 | 0.004 | 43.97 | 56.73 | 49.40 | 26.45 | 87.74 | 74.42 | 57.50 |
| 979 | 3285.6 | 9.5 | 2.003E−07 | 20.05 | 55.99 | 59.46 | 45.31 | 41.38 | 31.72 | 31.72 |
| 992 | 29869.1 | 5.51 | 8.784E−07 | 15.55 | 41.44 | 34.51 | 32.12 | 57.94 | 38.11 | 39.05 |

N/A: No match available in public information databases, pI: Isoelectric pH, MW: molecular weight
*Average Normalized Density × 100,000

Mass spectrometric identification of differentially expressed proteins in chagasic plasma. To discover maximally altered proteins that are of significance in characterizing the disease state and severity, 146 protein spots were submitted to MALDI-TOF MS/MS for protein identification. The protein spots for sequencing were chosen based upon their maximal differential expression in infected and/or treated groups. The MS and MS/MS spectral data were submitted to the UniProt rat proteome database for identification of proteins, and then homology searches were conducted against NCBI and SwissProt databases to validate protein identity. These analyses identified with high probability (MASCOT score>80%) 110 of the 146 proteins submitted for sequencing. Of the 110 differentially expressed identified proteins, 31 proteins were identified to be similar proteins resulting in 79 unique proteins (Tables 1a-1b). Among these differentially expressed proteins identified by mass spectrometry, 67 (65+2) appeared to be up regulated in infected rodents, and PBN normalized the expression of 16 of the proteins that were otherwise dysregulated in acute plasma. A comparison of plasma profiles between normal and infected rats revealed that 33 of the proteins were consistently up regulated during the acute infection and chronic disease phases, and hence may potentially be useful for diagnosis of Chagas disease. Two of the protein spots were strictly associated with the acute phase and 21 with the chronic phase and these are potentially useful for diagnosing disease states. When the effect of PBN and BZ treatment on the proteome profile was analyzed, 28 and 2 proteins, respectively, were stimulated in chronic chagasic plasma in a treatment-specific manner, and the increased plasma levels of 14 proteins in chronic rats was not altered by PBN and/or BZ treatment. Instead PBN+BZ treatment resulted in the altered expression of 61 (39+22) proteins, of which 22 were also altered in plasma of BZ-treated rats. Together, these results confirm that the plasma profile is altered by parasite persistence and oxidative stress, and treatment with PBN and BZ, known to control oxidative damage and inhibit parasite persistence, respectively (Wen et al., (2010) J Am Coll Cardiol, 55, 2499-2508), achieves its beneficial effects by normalizing the expression of disease-associated proteins and up regulation of other proteins.

Sub-cellular location and functional characterization. The differentially expressed protein datasets (Tables 1a-1b) were submitted to IPA and UniProt for identifying cellular localization and function analysis. As expected for plasma, >65% of the differentially expressed proteins were extracellular. Detection of the increased release of proteins belonging to cellular compartments, including the cell surface (3%), cytoplasm (6%), endoplasmic reticulum (2%), endosome (3%), membrane (13%) and nucleus (8%) in chagasic plasma, indicated that T. cruzi-induced cellular events and cell injury/cell death, and the resultant release of intracellular proteins, at least partially, contribute to disease-specific differential plasma proteomes.

Following a functional analysis of T. cruzi-stimulated proteins, a maximal number of the proteins belonged to the acute response/inflammation category (66%), of which >50% were normalized to control levels in the plasma of PBN-treated/infected rats. Presented are the top three networks with a score [−log(p-value)] of 25 or greater to which maximal number of the differentially expressed proteins identified in chagasic plasma (bolded) were associated with individual networks. Associated network analysis allowed classification of the differentially expressed proteins into three networks (Table 2), with the maximal number assigned to the antigen presentation, humoral immune response, and inflammatory response networks (19 proteins), followed by the lipid metabolism, molecular transport, small molecular biochemistry networks (17 proteins), and finally, the cellular development, free radical scavenging, molecular transport networks (14 proteins). In addition, diseases and disorders analysis by IPA also associated a maximal number of differentially expressed proteins to the acute phase response signaling pathway (13 proteins). Focus molecules are the number of differentially expressed plasma proteins associated with an individual network. Further IPA analysis of identified proteins to determine the correlation with heart diseases provided evidence that the differential plasma proteome was indicative of myocardial infarction, contractile dysfunction, hypertrophy and fibrosis, inflammation and tissue damage, and pulmonary embolism and hypertension in chagasic rats.

TABLE 2
IPA network analysis of differentially expressed plasma proteins in Chagasic rats

| | | | Focus | |
|---|---|---|---|---|
| ID | Molecules in Network | Score | Molecules | Top Functions |
| 1 | Actin, ALB, APOA1, APOA4, APOE, APOH, | 41 | 19 | Antigen |
| | C3, CFB, CFH, CFHR1, CFP, CHD4, | | | Presentation, |
| | Cytokeratin, DSTN, FGB, Fibrinogen, GSN, | | | Humoral Immune |
| | HPX, IgG, KRT1, KRT3, KRT4, KRT10, KRT12, | | | Response, |
| | KRT13, KRT23, KRT6B, PLG, SERPINF, | | | Inflammatory |
| | SERPINF1, SETX, TF, TMPRSS6, TRY6, VIM | | | Response |
| 2 | AFM, APP, BRPF3, C4BPA, CACNA1D, | 36 | 17 | Lipid Metabolism, |
| | COL25A1, DLGAP2, GAB1/2, GC, GRB2, | | | Molecular Transport, |
| | heparin, INSR, KCNMA1, KNG1 (includes | | | Small Molecule |
| | EG:16644), Met dimer, MUG1, MYLPF, | | | Biochemistry |
| | MY05A, MY05B, MY05C, PFKFB2, | | | |
| | PIK3AP1, PIK3R1, PLA2G2D, PLA2G2E, PRPH, | | | |
| | PZP, RAI14, SCG2, SERPINC1, SLC23A1, | | | |
| | SNX8, VPS13A, YWHAZ, ZNF32 | | | |
| 3 | ATG4C.BFAR, C10RF25, CASP8, | 29 | 14 | Cellular Development, |
| | CEBPB, CEP350, CIDEC, CRAT, DSCR3, | | | Free Radical |
| | ESR1, GNAQ, GPT, HNF4A, IDH3A, ITIH4, | | | Scavenging, Molecular |
| | LGMN, MINA, MYC, PEPD, PPARG, | | | Transport |
| | PUS1, PU S3, SCD2, SERPINA1, | | | |
| | SLC25A19, SQRDL (includes EG:58472), | | | |
| | SRPRB, SYMPK, TMEM176A, | | | |
| | TMEM176B, TRUB2, TTC37, UMPS, | | | |
| | YME1L1, ZNHIT6 | | | |

PBN, an antioxidant, controlled the pathologic oxidative tissue injury and mitochondrial dysfunction induced by T. cruzi, and co-treatment of rats with PBN and anti-parasite drug (BZ) was most effective in preserving the cardiac function in chronically infected chagasic hearts. The plasma proteome profile of chagasic rats showed that the recovery of heart function in PBN- and PBN/BZ-treated chagasic rats was associated with normalization of expression of the inflammation- and tissue remodeling-related proteins. Simultaneously, the plasma detection of other proteins was increased by PBN (e.g., SERPNAI, KRT1, SRPRB, Igh-1a, SroTP, TF, FGB, CDKSRAP2, Itih4 and PLG) and BZ (e.g. IDH3A, ALI3, MUG1, CFH. CEP350, COG1, AFM, ZFP37, ES2, ERCC4, SERPINC1, APOH, SPI-1, APOA4, SYMPK, RBM15B, and CHD4) treatment of infected rats. These data supports the concept that acute response and inflammation play an important role in Chagas disease development, and recovery of heart function by PBN/BZ treatment was reflected by the normalization of the plasma profile of immune and tissue remodeling responses in infected rats.

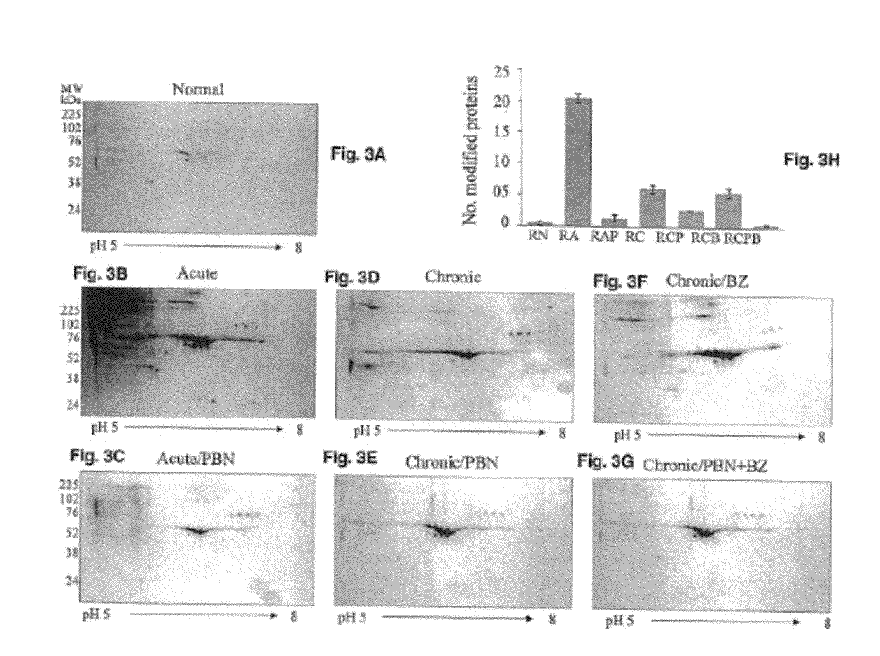

Carbonyl proteome profile in Chagas disease. Western blotting was performed to identify the plasma proteins that are sensitive to oxidative stress during Chagas disease. For this, protein-loaded 1-D IPG strips were incubated with DNPH to derivatize the carbonyl proteins, and after 2^{nd}-dimension gel electrophoresis, immuno-blotting was carried out by using anti-DNP antibody. FIGS. 3A-3G are the representative images of plasma carbonyl-proteome profiles in acute and chronic chagasic conditions, and the impact of PBN and BZ treatment on disease-related carbonyl proteomes. Overall, 115, 28, 34, 15, 31, and 12 protein spots were carbonylated in RA, RAP, RC, RCP, RCB and RCPB groups (FIG. 3H). These data clearly showed that plasma proteins were exposed to acute oxidative stress (FIG. 3B), and their oxidation was significantly diminished by PBN treatment (FIG. 3C).

At the chronic infection stage, the total number of carbonyl-proteins was less than that noted in the acute stage. It is, however, important that PBN treatment (FIGS. 3E and 3G), but not BZ treatment (FIG. 3F), ameliorated the protein carbonylation in chronic chagasic plasma. Comparative analysis of signal densities from SYPRO Ruby-stained gels (FIG. 1) and WB images (FIG. 3) that some proteins in the plasma of infected rodents (e.g. spot 115) were decreased in expression, but increased in carbonylation, while others (e.g. spot #184, 328, 355, 385, 389 and 766) were increased in expression as well as carbonylation. Upon PBN treatment, carbonylation of several of the proteins was ameliorated, either partially (e.g. spot #355, 389, 592, 604, 606) or completely (e.g. spot #73, 77, 115, 123, 153, 184, 207, 299, 328, 359, 385, 733, 766). Nine protein spots (299, 328, 385, 733, 766, 796, 906, 908, 948) were distinctly modified in acute plasma, and 15 proteins (spot #73, 77, 115, 123, 153, 184, 207, 355, 359, 389, 592, 604, 606, 614, and 1081) were modified throughout the course of infection and disease development. IPA analysis for cellular location revealed 56% of the proteins oxidatively modified in an infected host were extracellular. IPA analysis of biological response networks revealed a majority of the modified proteins (42%) represented host immunity to infection (i.e., acute phase response, complement activation and inflammatory response, response to cytokine stimulus), followed by protein transport (19%), tissue remodeling and metabolism (10%), and DNA catabolic process and myosin complex function (5% each). These data suggested that oxidative stress affects the expression level (and possibly function) of the proteins associated with inflammation and protein transport during T. cruzi infection.

Validation of differentially expressed proteins in Chagas disease. To validate the T. cruzi-induced differential expression of plasma proteins identified by 2D-GE/mass spectrometry, WB was performed with a protein-specific antibody. Plasma proteins were resolved by SDS-PAGE, and probed with antibodies to detect the expression level of GSN, MYL2, and VIM (FIGS. 4A-4D). Anti-GSN, anti-MYL2 and anti-VIM antibodies exhibited strong reactivity for 93 kDa, 20 kDa, and 57 kDa bands, respectively, in acute and chronic plasma (FIGS. 4A-4C). The enhanced plasma levels of GSN, MYL2, and VIM in chronically infected rats were normalized to the control level by PBN/BZ treatment of infected rats. Coomassie staining of the gels showed equal loading of all protein samples (FIG. 4D). These data verified the identity of GSN, MYL2 and VIM in chagasic plasma and validated the identification of proteins by mass spectrometry.

To investigate if the selected proteins exhibit disease-specific expression patterns in human patients and are useful biomarkers of the Chagas disease state, the plasma proteins from clinically characterized chagasic patients were resolved and WB was performed as above. Criteria for characterization of seropositive chagasic subjects exhibiting variable degrees of cardiac disease (CD0-CD3), seronegative cardiomyopathy patients of other etiologies; and seronegative, healthy individuals exhibiting no history or clinical symptom of cardiac disease have been described previously. Low levels of GSN and VIM and no MYL2 were detected in the plasma of normal healthy controls. In comparison, GSN, MYL2, and VIM were significantly increased in plasma from chagasic patients (FIG. 5A).

The detection of GSN, MYL2, and VIM in plasma from chagasic patients correlated directly with the disease stage, with a moderate increase in patients at the CD0-CD1 stage, while a maximal increase was noted in patients at the CD2/CD3 stage (FIGS. 5B and 5C). A moderate increase is defined as greater than a 40% increase and less than a 200% increase in levels relative to normal levels (i.e., the average levels measured in a subject of the same species that is not infected with T. cruzi). A maximal increase is defined as an increase of 200% (2-fold) or greater in measured levels relative to normal levels. Seronegative patients with cardiac symptoms due to other etiologies exhibited a marginal, but not statistically significant, increase in GSN, and no increase in MYL2 and VIM levels in sera when compared to these levels in normal controls (data not shown). These data validate the experimental results (FIGS. 4A-4D) and suggest that the plasma levels of GSN, MYL2, and VIM are useful indicators of disease and disease severity in human chagasic patients.

Analysis of serum proteomic signature of human chagasic patients for the identification of additional protein biomarkers of disease were conducted. The inventor employed IgY LC10 affinity chromatography in conjunction with ProteomeLab PF2D, two-dimensional gel-electrophoresis and western-blotting with anti-DNP antibody to resolve the proteome and oxidative-proteome signature of high-abundance and low-abundance serum proteins in chagasic and other cardiomyopathy patients, and MALDI-TOF-MS/MS approach to identify the disease-associated proteins. These studies identified a total of 80 and 13 differentially expressed proteins in chagasic and other cardiomyopathy patients, respectively. The extent of oxidative stress-induced carbonyl modifications of the differentially expressed proteins (n=26) was increased and coupled with a depression of antioxidant proteins in chagasic patients. Functional annotation of the top networks developed by Ingenuity Pathway Analysis of proteome database identified dysregulation of inflammation/acute phase response signaling and prostaglandins/arachidonic acid associated lipid metabolism in chagasic patients.

Overlay of the major networks identified prothrombin and plasminogen at a nodal position with connectivity to proteome signature indicative of heart disease (i.e. thrombosis, angiogenesis, vasodilation and increased permeability of blood vessels), and inflammatory responses (e.g., platelet aggregation, complement activation, phagocytes activation and migration). The detection of cardiac proteins (myosin light chain-2, myosin heavy chain-11) and increased levels of vinculin and plasminogen provided a comprehensive set of biomarkers of cardiac injury and development of clinical Chagas disease. These results provide an impetus for biomarker validation in large cohorts of clinically characterized chagasic patients.

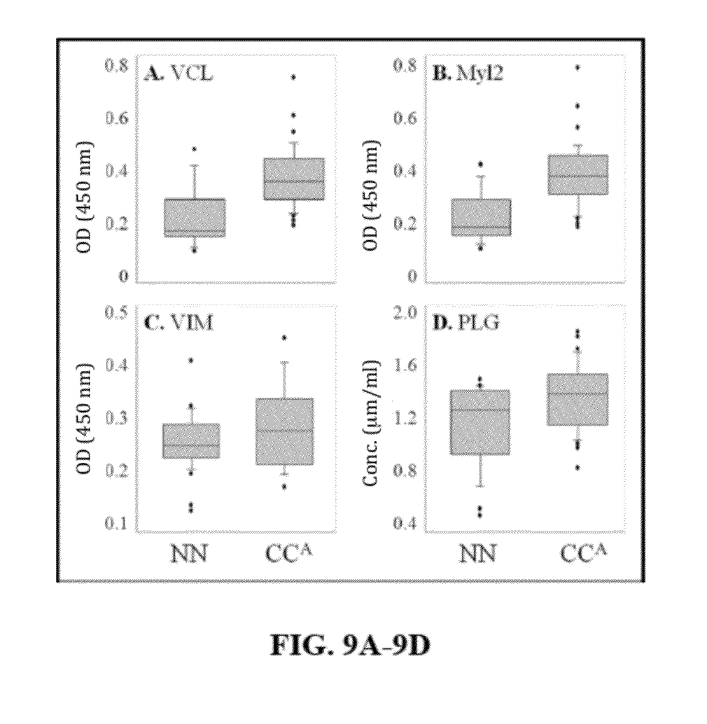

FIG. 9 shows the studies that were conducted to validate the expression profile of selected proteins in chagasic sera. Whole sera samples from normal (n=20) and chagasic (n=35) subjects were subjected to ELISA analysis by using antibodies against vinculin (VCL) (FIG. 9A), myosin light chain 2 (cardiac specific, MYL2) (FIG. 9B), vimentin (VIM) (FIG. 9C), and plasminogen (PLG) (FIG. 9D). Standard deviation for triplicate observations for each sample was <12%. These data exhibited a substantially higher level of VCL, MYL2, and PLG in seropositive chagasic patients compared to normal healthy controls (p<0.001). The inventor noticed ˜51% of the chagasic patients exhibited VCL levels significantly higher than the meanₛₑᵣₒₚₒₛᵢₜᵢᵥₑ values (mean OD_{450 nm}: 0.372±0.114 versus 0.186±0.053, range: 0.240-0.499 versus 0.101-0.412, seropositive versus seronegative, p<0.001). Likewise, 56% of the chagasic patients exhibited a significantly higher level of MYL2 than the meanₛₑᵣₒₚₒₛᵢₜᵢᵥₑ value (mean OD_{450 nm}: 0.329±0.101 versus 0.173±0.0181; range: 0.121-0.332 versus 0.204-0.423; seropositive versus seronegative, p<0.0001). Further, sera level of PLG was significantly increased in 32% of the chagasic patients (mean value: 216±33 versus 188±40; range: 147-280 versus 100-220 μg/ml; seropositive versus seronegative, p<0.0001). The sera levels of VIM were increased in 10% of the seropositive individuals as compared to those in normal healthy controls (mean OD_{450 nm}: 0.329±0.101 versus 0.173±0.0181; range: 0.204-0.423 versus 0.121-0.332; seropositive versus seronegative); however, these results were not statistically significant. Seronegative patients with cardiac symptoms due to other etiologies exhibited a marginal, but not statistically significant, increase in VCL, and no increase in MYL2 and VIM levels in sera when compared to these levels in normal controls (data not shown). Together, these data validate the PF2D/mass spectrometry results and lead us to suggest that the sera levels of VCL, MYL2, and PLG will be useful indicators of disease and disease severity in human chagasic patients.

The practice of the present invention will employ, unless otherwise indicated, conventional methods of chemistry, biochemistry, molecular biology, immunology, and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. Weir and C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, Wiley & Sons); Molecular Biology Techniques: An Intensive Laboratory Course (Ream et al., eds., 1998, Academic Press); PCR (Introduction to Biotechniques Series), 2nd ed. (Newton & Graham eds., 1997, Springer Verlag); Peters and Dalrymple, Fields Virology, 2nd ed., Fields et al. (eds.) (B.N. Raven Press, New York, N.Y.). All references cited in the description are incorporated herein by reference.

1. A method of determining the presence or severity of Chagas disease comprising the step of measuring the levels of at least one protein selected from the group consisting of vimentin (VIM), gelsolin (GSN), myosin light chain 2 (MYL2), myosin heavy chain 11 (MYH11), vinculin (VCL), and plasminogen (PLG) in a test sample from a subject, wherein an elevated level of the one or proteins compared to a control sample uninfected by T. cruzi indicates that the subject has Chagas disease.

2. The method of claim 1, wherein the levels of one or more of VCL, MYL2, or PLG is measured.

3. The method of claim 1, wherein the subject is a human.

4. The method of claim 1, wherein the test sample is a tissue sample, a plasma sample, or a blood sample.

5. The method of claim 1, wherein the test sample is a plasma sample.

6. The method of claim 1, wherein the levels of protein are measured by a Western blot assay, an ELISA assay, an immunofluorescence assay, an immunoprecipitation assay, a radioimmunoassay, or combinations thereof.

7. The method of claim 1, wherein the elevated level of a protein is greater than 40% and less that 200% relative to normal levels.

8. The method of claim 1, wherein the elevated level of a protein is greater than or equal to 200% relative to normal levels.

9. A method of treating Chagas disease comprising the steps of:
(a) determining the presence or severity of Chagas disease as described in claim 1; and
(b) administering an agent to treat parasitic infection, cardiomyopathy, or both to a subject identified as having Chagas disease.

10. A serodiagnostic kit for determining whether a subject is infected with Trypanosoma cruzi, said kit comprising:
(a) an antibody directed against vimentin, gelsolin, myosin light chain 2, myosin heavy chain 11, vinculin or plasminogen, wherein said antibody is linked to a reporter molecule;
(b) a buffer; and
(c) a reagent for detection of the reporter molecule.

11. The kit of claim 10, wherein the reporter molecule is selected from the group consisting of luciferase, horseradish peroxidase, P-galactosidase, and fluorescent labels.
